SEQUENCE LISTING (62 sequences)

SEQ ID NO: 1 - DNA, 340 bp, Homo sapiens
gatgttgtgatgactcagtctccactctccctgcccgtcacccttggacagccggcctccatctcctgcaggtctagtcaaagcctcgtatacagtgatggaaacacctacttgaattggtttcagcagaggccaggccaatctccaaggcgcctaatttataaggtttctaaccgggactctggggtcccagacagattcagcggcagtgggtcaggcactgatttcacactgaaaatcagcagggtggaggctgaggatgttggggtttattactgcatgcaaggtacacactggcctccgtggacgttcggccaagggaccaaggtggaaatcaaac

SEQ ID NO: 2 - protein, 113 aa, Homo sapiens
DVVMTQSPLSLPVTLGQPASISCRSSQSLVYSDGNTYLNWFQQRPGQSPRRLIYKVSNRDSGVPDRFSGSGSGTDFTLKISRVEAEDVGVYYCMQGTHWPPWTFGQGTKVEIK

SEQ ID NO: 3 - DNA, 340 bp, Homo sapiens
gatattgtgatgacccagactccactctctctgtccgtcacccctggacagccggcctccatctcctgcaagtctagtcagagcctcctgcatagtgatggaaagacctatttgtattggtacctgcagaagccaggccagtctccacagctcctaatctatgaagtttccagccggttctctggagtgccagataggttcagtggcagcgggtcagggacagatttcacactgaaaatcagccgggtggaggctgaggatgttggggtttattactgcatgcaaggtatacaccttcctccgtggacgttcggccaagggaccaaggtggaaatcaaac

SEQ ID NO: 4 - protein, 113 aa, Homo sapiens
DIVMTQTPLSLSVTPGQPASISCKSSQSLLHSDGKTYLYWYLQKPGQSPQLLIYEVSSRFSGVPDRFSGSGSGTDFTLKISRVEAEDVGVYYCMQGIHLPPWTFGQGTKVEIK

SEQ ID NO: 5 - DNA, 337 bp, Homo sapiens
gatattgtgatgacccagactccactctctctgtccgtcacccctggacagccggcctccatctcctgcaagtctagtcagagcctcctgcatagtgatggaaagacctatttgtattggtacctgcagaagccaggccagtctccacagctcctaatctatgaagtttccagccggttctctggagtgccagataggttcagtggcagcgggtcagggacagatttcacactgaaaatcagccgggtggaggctgaggatgttggggtttattactgcatgcaaggtatacactttcctcagacgttcggtggagggaccaaggtggaaatcaaac

SEQ ID NO: 6 - protein, 112 aa, Homo sapiens
DIVMTQTPLSLSVTPGQPASISCKSSQSLLHSDGKTYLYWYLQKPGQSPQLLIYEVSSRFSGVPDRFSGSGSGTDFTLKISRVEAEDVGVYYCMQGIHFPQTFGGGTKVEIK

SEQ ID NO: 7 - DNA, 322 bp, Homo sapiens
gacatccagatgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataatagttacccttggacgttcggccaagggaccaaggtggaaatcaaac

SEQ ID NO: 8 - protein, 107 aa, Homo sapiens
DIQMTQSPSSLSASVGDRVTITCRASQGIRNDLGWYQQKPGKAPKRLIYAASSLQSGVPSRFSGSGSGTEFTLTISSLQPEDFATYYCLQHNSYPWTFGQGTKVEIK

SEQ ID NO: 9 - DNA, 322 bp, Homo sapiens
aacatccagatgacccagtctccatctgccatgtctgcatctgtaggagacagagtcaccatcacttgtcgggcgaggcagggcattagcaattatttagcctggtttcagcagaaaccagggaaagtccctaagcacctgatctatgctgcatccagtttgcaaagtggggtcccatcaaggttcagcggcagtggatctgggacagaattcactctcacaatcagcagcctgcagcctgaagattttgcaacttattactgtctacagcataatagttacccttggacgttcggccaagggaccaaggtggaaatcaaac

SEQ ID NO: 10 - protein, 107 aa, Homo sapiens
NIQMTQSPSAMSASVGDRVTITCRARQGISNYLAWFQQKPGKVPKHLIYAASSLQSGVPSRFSGSGSGTEFTLTISSLQPEDFATYYCLQHNSYPWTFGQGTKVEIK

SEQ ID NO: 11 - DNA, 337 bp, Homo sapiens
gatgttgtgatgactcagtctccactctccctgcccgtcacccttggacagccggcctccatctcctgcaggtctagtcaaagcctcgtatacagtgatggaaacacctacttgaattggtttcagcagaggccaggccaatctccaaggcgcctaatttataaggtttctaactgggactctggggtcccagacagattcagcggcagtgggtcaggcactgatttcacactgaaaatcagcagggtggaggctgaggatgttggggtttattactgcatgcaaggtacacactggccgtggacgttcggccaagggaccaaggtggaaatcaaac

SEQ ID NO: 12 - protein, 112 aa, Homo sapiens
DVVMTQSPLSLPVTLGQPASISCRSSQSLVYSDGNTYLNWFQQRPGQSPRRLIYKVSNWDSGVPDRFSGSGSGTDFTLKISRVEAEDVGVYYCMQGTHWPWTFGQGTKVEIK

SEQ ID NO: 13 - DNA, 340 bp, Homo sapiens
gatattgtgatgacccagactccactctctctgtccgtcacccctggacagccggcctccatctcctgcaagtctagtcagagcctcctgcatagtgatggaaagacctatttgtattggtacctgcagaagccaggccagtctccacagctcctgatctatgaagtttccaaccggttctctggagtgccagataggttcagtggcagcgggtcagggacagatttcacactgaaaatcagccgggtggaggctgaggatgttggggtttattactgcatgcaaagtatacagcttcctccgtggacgttcggccaagggaccaaggtggaaatcaaac

SEQ ID NO: 14 - protein, 113 aa, Homo sapiens
DIVMTQTPLSLSVTPGQPASISCKSSQSLLHSDGKTYLYWYLQKPGQSPQLLIYEVSNRFSGVPDRFSGSGSGTDFTLKISRVEAEDVGVYYCMQSIQLPPWTFGQGTKVEIK

SEQ ID NO: 15 - DNA, 337 bp, Homo sapiens
gatattgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactccgtggacgttcggccaagggaccaaggtggaaatcaaac

SEQ ID NO: 16 - protein, 112 aa, Homo sapiens
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPWTFGQGTKVEIK

SEQ ID NO: 17 - DNA, 337 bp, Homo sapiens
gatattgtgatgacccagactccactctcctcacctgtcacccttggacagccggcctccatctcctgcaggtctagtcaaagcctcgtacacagtgatggaaacacctacttgagttggcttcagcagaggccaggccagcctccaagactcctaatttataagatttctaaccggttctctggggtcccagacagattcagtggcagtggggcagggacagatttcacactgaaaatcagcagggtggaagctgaggatgtcggggtttattactgcatgcaagctacacaatttccgtggacgttcggccaagggaccaaggtggaaatcaaac

SEQ ID NO: 18 - protein, 112 aa, Homo sapiens
DIVMTQTPLSSPVTLGQPASISCRSSQSLVHSDGNTYLSWLQQRPGQPPRLLIYKISNRFSGVPDRFSGSGAGTDFTLKISRVEAEDVGVYYCMQATQFPWTFGQGTKVEIK

SEQ ID NO: 19 - DNA, 322 bp, Homo sapiens
gaaattgtgttgacacagtctccagccaccctgtctttgtctccaggggaaagagccaccctctcctgcagggccagtcagggtgttagcagctacttagcctggtaccagcagaaacctggccaggctcccaggctcctcatctatgatgcatccaacagggccactggcatcccagccaggttcagtggcagtgggcctgggacagacttcactctcaccatcagcagcctagagcctgaagattttgcagtttattactgtcagcagcgtagcaactggcagtggacgttcggccaagggaccaaggtggaaatcaaac

SEQ ID NO: 20 - protein, 107 aa, Homo sapiens
EIVLTQSPATLSLSPGERATLSCRASQGVSSYLAWYQQKPGQAPRLLIYDASNRATGIPARFSGSGPGTDFTLTISSLEPEDFAVYYCQQRSNWQWTFGQGTKVEIK

SEQ ID NO: 21 - DNA, 322 bp, Homo sapiens
gaaacgacactcacgcagtctccagcattcatgtcagcgactccaggagacaaagtcaacatctcctgcaaagccagccaagacattgatgatgatatgaactggtaccaacagaaaccaggagaagctgctattttcattattcaagaagctactactctcgttcctggaatcccacctcgattcagtggcagcgggtatggaacagattttaccctcacaattaataacatagaatctgaggatgctgcatattacttctgtctacaacatgataatttcccgtggacgttcggccaagggaccaaggtggaaatcaaac

SEQ ID NO: 22 - protein, 107 aa, Homo sapiens
ETTLTQSPAFMSATPGDKVNISCKASQDIDDDMNWYQQKPGEAAIFIIQEATTLVPGIPPRFSGSGYGTDFTLTINNIESEDAAYYFCLQHDNFPWTFGQGTKVEIK

SEQ ID NO: 23 - DNA, 322 bp, Homo sapiens
gaaattgtgctgactcagtctccagactttcagtctgtgactccaaaggagaaagtcaccatcacctgccgggccagtcagagcattggtagtagcttacactggtaccagcagaaaccagatcagtctccaaagctcctcatcaagtatgcttcccagtccttctcaggggtcccctcgaggttcagtggcagtggatctgggacagatttcaccctcaccatcaatagcctggaagctgaagatgctgcaacgtattactgtcatcagagtagtagtttaccgtggacgttcggccaagggaccaaggtggaaatcaaac

SEQ ID NO: 24 - protein, 107 aa, Homo sapiens
EIVLTQSPDFQSVTPKEKVTITCRASQSIGSSLHWYQQKPDQSPKLLIKYASQSFSGVPSRFSGSGSGTDFTLTINSLEAEDAATYYCHQSSSLPWTFGQGTKVEIK

SEQ ID NO: 25 - DNA, 322 bp, Homo sapiens; identical to SEQ ID NO: 23

SEQ ID NO: 26 - protein, 107 aa, Homo sapiens; identical to SEQ ID NO: 24

SEQ ID NO: 27 - DNA, 325 bp, Homo sapiens
gatgttgtgatgacacagtctccagctttcctctctgtgactccaggggagaaagtcaccatcacctgccaggccagtgaaggcattggcaactacttatactggtaccagcagaaaccagatcaagccccaaagctcctcatcaagtatgcttcccagtccatctcaggggtcccctcgaggttcagtggcagtggatctgggacagatttcacctttaccatcagtagcctggaagctgaagatgctgcaacatattactgtcagcagggcaataagcaccctcagtggacgttcggccaagggaccaaggtggaaatcaaac

SEQ ID NO: 28 - protein, 108 aa, Homo sapiens
DVVMTQSPAFLSVTPGEKVTITCQASEGIGNYLYWYQQKPDQAPKLLIKYASQSISGVPSRFSGSGSGTDFTFTISSLEAEDAATYYCQQGNKHPQWTFGQGTKVEIK

SEQ ID NO: 29 - protein, 95 aa, Homo sapiens
DIQMTQSPSSLSASVGDRVTITCRASQSISSYLNWYQQKPGKAPKLLIYAASSLQSGVPSRFSGSGSGTDFTLTISSLQPEDFATYYCQQSYSTP

SEQ ID NO: 30 - protein, 95 aa, Homo sapiens
DIQMTQSPSSLSASVGDRVTITCQASQDISNYLNWYQQKPGKAPKLLIYDASNLETGVPSRFSGSGSGTDFTFTISSLQPEDIATYYCQQYDNLP

SEQ ID NO: 31 - protein, 95 aa, Homo sapiens
DIQMTQSPSSLSASVGDRVTITCRASQGISNYLAWYQQKPGKVPKLLIYAASTLQSGVPSRFSGSGSGTDFTLTISSLQPEDVATYYCQKYNSAP

SEQ ID NO: 32 - protein, 95 aa, Homo sapiens
DIQMTQSPSSLSASVGDRVTITCRASQGIRNDLGWYQQKPGKAPKRLIYAASSLQSGVPSRFSGSGSGTEFTLTISSLQPEDFATYYCLQHNSYP

SEQ ID NO: 33 - protein, 95 aa, Homo sapiens
NIQMTQSPSAMSASVGDRVTITCRARQGISNYLAWFQQKPGKVPKHLIYAASSLQSGVPSRFSGSGSGTEFTLTISSLQPEDFATYYCLQHNSYP

SEQ ID NO: 34 - protein, 95 aa, Homo sapiens
DIQMTQSPSSLSASVGDRVTITCRASQGISNYLAWFQQKPGKAPKSLIYAASSLQSGVPSRFSGSGSGTDFTLTISSLQPEDFATYYCQQYNSYP

SEQ ID NO: 35 - protein, 95 aa, Homo sapiens
DIQMTQSPSSLSASVGDRVTITCRASQGISSWLAWYQQKPEKAPKSLIYAASSLQSGVPSRFSGSGSGTDFTLTISSLQPEDFATYYCQQYNSYP

SEQ ID NO: 36 - protein, 95 aa, Homo sapiens
AIQLTQSPSSLSASVGDRVTITCRASQGISSALAWYQQKPGKAPKLLIYDASSLESGVPSRFSGSGSGTDFTLTISSLQPEDFATYYCQQFNSYP

SEQ ID NO: 37 - protein, 95 aa, Homo sapiens
DIQMTQSPSSVSASVGDRVTITCRASQGISSWLAWYQQKPGKAPKLLIYAASSLQSGVPSRFSGSGSGTDFTLTISSLQPEDFATYYCQQANSFP

SEQ ID NO: 38 - protein, 95 aa, Homo sapiens
DIQLTQSPSFLSASVGDRVTITCRASQGISSYLAWYQQKPGKAPKLLIYAASTLQSGVPSRFSGSGSGTEFTLTISSLQPEDFATYYCQQLNSYP

SEQ ID NO: 39 - protein, 95 aa, Homo sapiens
AIRMTQSPFSLSASVGDRVTITCWASQGISSYLAWYQQKPAKAPKLFIYYASSLQSGVPSRFSGSGSGTDYTLTISSLQPEDFATYYCQQYYSTP

SEQ ID NO: 40 - protein, 95 aa, Homo sapiens
AIRMTQSPSSFSASTGDRVTITCRASQGISSYLAWYQQKPGKAPKLLIYAASTLQSGVPSRFSGSGSGTDFTLTISCLQSEDFATYYCQQYYSYP

SEQ ID NO: 41 - protein, 95 aa, Homo sapiens
VIWMTQSPSLLSASTGDRVTISCRMSQGISSYLAWYQQKPGKAPELLIYAASTLQSGVPSRFSGSGSGTDFTLTISCLQSEDFATYYCQQYYSFP

SEQ ID NO: 42 - protein, 95 aa, Homo sapiens
AIQMTQSPSSLSASVGDRVTITCRASQGIRNDLGWYQQKPGKAPKLLIYAASSLQSGVPSRFSGSGSGTDFTLTISSLQPEDFATYYCLQDYNYP

SEQ ID NO: 43 - protein, 95 aa, Homo sapiens
DIQMTQSPSTLSASVGDRVTITCRASQSISSWLAWYQQKPGKAPKLLIYDASSLESGVPSRFSGSGSGTEFTLTISSLQPDDFATYYCQQYNSYS

SEQ ID NO: 44 - protein, 101 aa, Homo sapiens
DIVMTQTPLSLPVTPGEPASISCRSSQSLLDSDDGNTYLDWYLQKPGQSPQLLIYTLSYRASGVPDRFSGSGSGTDFTLKISRVEAEDVGVYYCMQRIEFP

SEQ ID NO: 45 - protein, 100 aa, Homo sapiens
DVVMTQSPLSLPVTLGQPASISCRSSQSLVYSDGNTYLNWFQQRPGQSPRRLIYKVSNRDSGVPDRFSGSGSGTDFTLKISRVEAEDVGVYYCMQGTHWP

SEQ ID NO: 46 - protein, 100 aa, Homo sapiens
DVVMTQSPLSLPVTLGQPASISCRSSQSLVYSDGNTYLNWFQQRPGQSPRRLIYKVSNWDSGVPDRFSGSGSGTDFTLKISRVEAEDVGVYYCMQGTHWP

SEQ ID NO: 47 - protein, 100 aa, Homo sapiens
DIVMTQTPLSLSVTPGQPASISCKSSQSLLHSDGKTYLYWYLQKPGQSPQLLIYEVSSRFSGVPDRFSGSGSGTDFTLKISRVEAEDVGVYYCMQGIHLP

SEQ ID NO: 48 - protein, 100 aa, Homo sapiens
DIVMTQTPLSLSVTPGQPASISCKSSQSLLHSDGKTYLYWYLQKPGQPPQLLIYEVSNRFSGVPDRFSGSGSGTDFTLKISRVEAEDVGVYYCMQSIQLP

SEQ ID NO: 49 - protein, 100 aa, Homo sapiens
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTP

SEQ ID NO: 50 - protein, 100 aa, Homo sapiens
DIVMTQTPLSSPVTLGQPASISCRSSQSLVHSDGNTYLSWLQQRPGQPPRLLIYKISNRFSGVPDRFSGSGAGTDFTLKISRVEAEDVGVYYCMQATQFP

SEQ ID NO: 51 - protein, 96 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSP

SEQ ID NO: 52 - protein, 96 aa, Homo sapiens
EIVLTQSPATLSLSPGERATLSCGASQSVSSSYLAWYQQKPGLAPRLLIYDASSRATGIPDRFSGSGSGTDFTLTISRLEPEDFAVYYCQQYGSSP

SEQ ID NO: 53 - protein, 95 aa, Homo sapiens
EIVMTQSPATLSVSPGERATLSCRASQSVSSNLAWYQQKPGQAPRLLIYGASTRATGIPARFSGSGSGTEFTLTISSLQSEDFAVYYCQQYNNWP

SEQ ID NO: 54 - protein, 95 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSNWP

SEQ ID NO: 55 - protein, 95 aa, Homo sapiens
EIVLTQSPATLSLSPGERATLSCRASQGVSSYLAWYQQKPGQAPRLLIYDASNRATGIPARFSGSGPGTDFTLTISSLEPEDFAVYYCQQRSNWH

SEQ ID NO: 56 - protein, 96 aa, Homo sapiens
EIVMTQSPATLSLSPGERATLSCRASQSVSSSYLSWYQQKPGQAPRLLIYGASTRATGIPARFSGSGSGTDFTLTISSLQPEDFAVYYCQQDYNLP

SEQ ID NO: 57 - protein, 101 aa, Homo sapiens
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTP

SEQ ID NO: 58 - protein, 95 aa, Homo sapiens
ETTLTQSPAFMSATPGDKVNISCKASQDIDDDMNWYQQKPGEAAIFIIQEATTLVPGIPPRFSGSGYGTDFTLTINNIESEDAAYYFCLQHDNFP

SEQ ID NO: 59 - protein, 95 aa, Homo sapiens
EIVLTQSPDFQSVTPKEKVTITCRASQSIGSSLHWYQQKPDQSPKLLIKYASQSFSGVPSRFSGSGSGTDFTLTINSLEAEDAATYYCHQSSSLP

SEQ ID NO: 60 - protein, 95 aa, Homo sapiens
DVVMTQSPAFLSVTPGEKVTITCQASEGIGNYLYWYQQKPDQAPKLLIKYASQSISGVPSRFSGSGSGTDFTFTISSLEAEDAATYYCQQGNKHP

SEQ ID NO: 61 - protein, 6 aa, Artificial Sequence
  Description of Artificial Sequence6xHIS six histidine linker, 6-histidine tag
HHHHHH

SEQ ID NO: 62 - protein, 200 aa, Artificial Sequence
  Description of Artificial Sequencepoly-Gly flexible linker
GGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGG

(19) United States
(12) Patent Application Publication
(10) Pub. No.: US 2006/0088883 A1
(43) Pub. Date: Apr. 27, 2006

(54) Recombinant catalytic polypeptides and their uses

(75) Inventors: Vaughn Smider, Alameda, CA (US); James W. Larrick, Woodside, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Integrigen, Inc., Novato, CA (US)

(21) Appl. No.: 11/239,308

(22) Filed: Sep. 28, 2005

Related U.S. Application Data
(63) Continuation of application No. 10/683,733, filed on Oct. 9, 2003, now abandoned.
(60) Provisional application No. 60/417,979, filed on Oct. 9, 2002.

(51) Int. Cl. C40B 40/10; C12N 9/00; C12P 21/06

(52) U.S. Cl. 435/7.1; 435/188.5; 435/68.1

(57) ABSTRACT

The present invention provides a recombinant catalytic polypeptide for cleaving a target protein, the nucleic acid encoding the recombinant catalytic polypeptide, a cell hosting the nucleic acid encoding the recombinant catalytic polypeptide, and a non-human transgenic mammal that is capable of producing a heterologous antibody with proteolytic activity. The invention also provides methods of cleaving a target protein using the recombinant catalytic polypeptides both in vitro and in vivo. The invention further provides a library of recombinant catalytic polypeptides with altered enzymatic activity and a method to alter enzymatic activity of the recombinant catalytic polypeptides.

### CROSS-REFERENCES TO RELATED APPLICATIONS

This application claims priority of U.S. provisional patent application Ser. No. 60/417,979, filed Oct. 10, 2002.

### BACKGROUND OF THE INVENTION

The present invention relates generally to the field of molecular biology and immunology, and relates specifically to recombinant catalytic polypeptides comprising two heterologous human antibody chains operably joined for specific cleavage of target proteins, as well as methods for the preparation of the recombinant polypeptides and their uses.

The present invention relates to catalytic antibodies. The earliest speculations that antibodies may possess catalytic activity date back half a century ago when it was suggested that if exposed to an antigen for a sufficiently long period, the immune system may develop catalytic antibodies (Woolley, A Study of Antimetabolites, p 82. Wiley, New York, 1952). Sequence homology between certain antibody light chain and serine proteases was later revealed, prompting inquiry into the possibility that some immunoglobulins may have proteolytic activity (Erhan and Greller, Nature, 251:353-355 (1974)). Several years later, antibodies with esterase activity were reported (Kohen et al., FEBS Letter, 111:427-431 (1980)). Further studies discovered antibodies capable of hydrolyzing peptides or proteins (see, e.g., Paul et al., Science, 244:1158-1162 (1989); Li et al., J. Immunol., 154:3328-3332 (1995)), hydrolyzing DNA (Shuster et al., Science, 256:665-667 (1992); Gololobov et al., Proc. Natl. Acad. Sci. USA, 92:254-257 (1995)), and with peroxidase activity (Paul, Mol. Biotechnol., 5:197-207 (1996)).

Catalytic antibodies can be isolated from the natural immune repertoire, but seem to be produced at an elevated level in various autoimmune disease states (Paul, supra). Analyses of catalytic antibody components have shown that enzymatic activity often resides in the light chains, and antibody light chains isolated from multiple myeloma patients frequently demonstrate proteolytic activity (Paul, supra).

Studies have provided evidence to connect proteolytic antibodies and serine proteases. Serine proteases are a large family of proteolytic enzymes that include the digestive enzymes, trypsin and chymotrypsin, components of the complement cascade and of the blood-clotting cascade, and enzymes that control the degradation and turnover of macromolecules of the extracellular matrix. They are so named because of the presence of a serine residue in the active catalytic site for protein cleavage. Serine proteases have a wide range of substrate specificities and diverse biological functions. Despite such diversity and often unrelated amino acid sequence, a common catalytic mechanism is shared among several sub-families of serine proteases through a very similar tertiary structure supported by a highly conserved catalytic triad of serine, histidine, and aspartate. The active site structure of one serine protease, subtilisin, is among the most studied and best understood.

There is strong indication of structural similarity at catalytic site between proteolytic antibodies and serine proteases. For example, it has been demonstrated that diisopropyl fluorophosphate, a serine protease inhibitor, strongly inhibited the catalytic activity of some proteolytic antibodies, whereas inhibitors of metalloproteases, acid proteases, and cysteine proteases had minimal effect, suggesting that such proteolytic antibodies have a catalytic mechanism similar to that of a serine protease (Paul et al., J. Bio. Chem., 256:16128-16134, (1991)). Molecular modeling of the light chain of an antibody capable of hydrolyzing vasoactive intestinal polypeptide (VIP, a 28-amino acid neuropeptide) further revealed an arrangement of Ser27a, His93, and Asp1 similar to the catalytic triad arrangement of a subfamily of serine proteases (Gao et al., J. Bio. Chem., 269:32389-32393 (1994)). Moreover, a substitution of alanine for any one of the three amino acid residues dramatically reduced the antibody's ability to hydrolyze VIP (Gao et al., J. Bio. Chem., 253:658-664 (1995)). Taken together, some proteolytic antibodies appear to utilize a serine protease-like mechanism for their catalytic activity.

While recent studies have provided better understanding as to the mechanism and regulation of catalytic antibodies (see, e.g., U.S. Pat. Nos. 5,658,753 and 6,235,714), the present invention takes a novel approach to create proteases with substrate specificities that do not exist in nature. Through somatic rearrangement, the mammalian immune system is capable of generating more than 10¹⁰ different antigen specificities, using only a limited number of germ line genes (Kuby, supra). In contrast, most known proteases or peptidases often target particular peptide bonds but can cleave a relatively broad spectrum of polypeptides without a high level of specificity for individual substrates. Combining a human antibody light chain that houses proteolytic activity with a heterologous human antibody heavy chain that provides polypeptide-binding specificity, the present invention provides a novel strategy of designing proteases that allows the specific hydrolysis of pre-selected target proteins without undesired effect on untargeted polypeptides. Given the vast number of antigen specificities the immune system can produce, as well as the virtually endless antigen specificities in vitro DNA technology can generate, potentially every protein can be specifically targeted for hydrolysis by a customized protease. This strategy will have profound implications in treatment and prevention of many diseases and conditions, where inappropriately elevated protein expression or the presence of an exogenous protein is known to contribute to the pathogenesis of such diseases or conditions.

### BRIEF SUMMARY OF THE INVENTION

In one aspect, the invention provides for recombinant catalytic polypeptides for cleaving target proteins. Each of the recombinant catalytic polypeptides comprises a human antibody light chain operably joined to a heterologous antibody heavy chain. The human antibody light chain has a serine protease dyad and endopeptidase activity, and the antibody heavy chain has a predetermined specificity for a target protein.

In some embodiments, the target proteins are selected from a group consisting of growth factors, cell surface receptors, cytokines, and immunoglobulins. In a preferred embodiment, the target protein is a vascular endothelial growth factor. In another preferred embodiment, the target protein is interferon γ. In another preferred embodiment, the target protein is TNF α. In another preferred embodiment, the target protein is a member of the IgE family. In another preferred embodiment, the target protein is a member of the EGF receptor family. In yet another preferred embodiment, the target protein is CD20. In other embodiments, the human antibody light chain has a serine protease triad. In other embodiments, the recombinant catalytic polypeptide is a single polypeptide chain that contains the human antibody light chain and the antibody heavy chain. In a preferred embodiment, the human antibody light chain comprises an amino acid sequence that has at least 80% identity to SEQ ID NO:2, 4, 6, 8, 10, 12, 14, 16, 18, 20, 22, 24, 26, or 28. In a more preferred embodiment, the human antibody light chain comprises an amino acid sequence that has at least 95% identity to SEQ ID NO:2, 4, 6, 8, 10, 12, 14, 16, 18, 20, 22, 24, 26, or 28. In a most preferred embodiment, the human antibody light chain comprises an amino acid sequence of SEQ ID NO:2, 4, 6, 8, 10, 12, 14, 16, 18,20, 22, 24, 26, or 28.

In another aspect, the invention provides for methods for cleaving target proteins. The methods typically comprise the step of contacting a target protein with a recombinant catalytic polypeptide under the conditions suitable for cleaving the target protein. The recombinant catalytic polypeptide comprises a human antibody light chain that is operably joined to a heterologous heavy chain. The antibody light chain has a serine protease dyad and endopeptidase activity, and the heavy chain has a predetermined activity for the target protein.

In some embodiments, the target proteins are selected from a group consisting of growth factors, cell surface receptors, cytokines, and immunoglobulins. In other embodiments, the human antibody light chain has a serine protease triad. In other embodiments, the recombinant catalytic polypeptide is a single polypeptide chain that contains the human antibody light chain and the antibody heavy chain. In a preferred embodiment, the human antibody light chain comprises an amino acid sequence that has at least 80% identity to SEQ ID NO:2, 4, 6, 8, 10, 12, 14, 16, 18, 20, 22, 24, 26, or 28. In a more preferred embodiment, the human antibody light chain comprises an amino acid sequence that has at least 95% identity to SEQ ID NO:2, 4, 6, 8, 10, 12, 14, 16, 18, 20, 22, 24, 26, or 28. In a most preferred embodiment, the human antibody light chain comprises an amino acid sequence of SEQ ID NO:2, 4, 6, 8, 10, 12, 14, 16, 18, 20, 22, 24, 26, or 28.

In another aspect, the invention provides for methods for altering the enzymatic activity of a recombinant catalytic polypeptide for cleaving a target protein. The methods typically comprise the steps of mutating at least one of the CDRs of an antibody heavy chain and determining mutations that altered in enzymatic activity of the polypeptide. The recombinant catalytic polypeptide comprises a human antibody light chain that is operably joined to a heterologous heavy chain. The antibody light chain has a serine protease dyad and endopeptidase activity, and the heavy chain has a predetermined activity for the target protein.

In some embodiments, an exonuclease is used in the step of mutating the CDRs of the antibody heavy chains. In other embodiments, the target proteins are selected from a group consisting of growth factors, cell surface receptors, cytokines, and immunoglobulins. In other embodiments, the human antibody light chain has a serine protease triad. In other embodiments, the recombinant catalytic polypeptide is a single polypeptide chain that contains the human antibody light chain and the antibody heavy chain. In a preferred embodiment, the human antibody light chain comprises an amino acid sequence that has at least 80% identity to SEQ ID NO:2, 4, 6, 8, 10, 12, 14, 16, 18, 20, 22, 24, 26, or 28. In a more preferred embodiment, the human antibody light chain comprises an amino acid sequence that has at least 95% identity to SEQ ID NO:2, 4, 6, 8, 10, 12, 14, 16, 18, 20, 22, 24, 26, or 28. In a most preferred embodiment, the human antibody light chain comprises an amino acid sequence of SEQ ID NO:2, 4, 6, 8, 10, 12, 14, 16, 18, 20, 22, 24, 26, or 28.

In another aspect, the invention provides for libraries of recombinant catalytic polypeptide members for cleaving target proteins. The library members typically comprise recombinant catalytic polypeptides, and have different CDRs in their respective heavy chains. Each recombinant catalytic polypeptide comprises a human antibody light chain that is operably joined to a heterologous heavy chain. The antibody light chain has a serine protease dyad and endopeptidase activity, and the heavy chain has a predetermined activity for the target protein.

In some embodiments, the target proteins are selected from a group consisting of growth factors, cell surface receptors, cytokines, and immunoglobulins. In other embodiments, the human antibody light chain has a serine protease triad. In other embodiments, the recombinant catalytic polypeptide is a single polypeptide chain that contains the human antibody light chain and the antibody heavy chain. In a preferred embodiment, the human antibody light chain comprises an amino acid sequence that has at least 80% identity to SEQ ID NO:2, 4, 6, 8,10,12,14, 16,18,20,22,24,26, or 28. In a more preferred embodiment, the human antibody light chain comprises an amino acid sequence that has at least 95% identity to SEQ ID NO:2, 4, 6, 8, 10, 12, 14, 16, 18, 20, 22, 24, 26, or 28. In a most preferred embodiment, the human antibody light chain comprises an amino acid sequence of SEQ ID NO:2, 4, 6, 8, 10, 12, 14, 16, 18, 20, 22, 24, 26, or 28. In some embodiments, the library is a phage display library. In other embodiments, the library is a ribosomal display library.

In another aspect, the invention provides for methods for cleaving target proteins in a mammal. The methods typically comprise the step of administering a recombinant catalytic polypeptide in an amount sufficient to lower the concentration of the target proteins in the mammal. The recombinant catalytic polypeptide comprises a human antibody light chain that is operably joined to a heterologous heavy chain. The antibody light chain has a serine protease dyad and endopeptidase activity, and the heavy chain has a predetermined activity for the target protein.

In some embodiments, the target proteins are selected from a group consisting of growth factors, cell surface receptors, cytokines, and immunoglobulins. In other embodiments, the human antibody light chain has a serine protease triad. In other embodiments, the recombinant catalytic polypeptide is a single polypeptide chain that contains the human antibody light chain and the antibody heavy chain. In a preferred embodiment, the human antibody light chain comprises an amino acid sequence that has at least 80% identity to SEQ ID NO:2, 4, 6, 8, 10, 12, 14, 16, 18, 20, 22, 24, 26, or 28. In a more preferred embodiment, the human antibody light chain comprises an amino acid sequence that has at least 95% identity to SEQ ID NO:2, 4, 6, 8, 10, 12, 14, 16, 18, 20, 22, 24, 26, or 28. In a most preferred embodiment, the human antibody light chain comprises an amino acid sequence of SEQ ID NO:2, 4, 6, 8, 10, 12, 14, 16, 18, 20, 22, 24, 26, or 28.

In another aspect, the invention provides for nucleic acids encoding recombinant catalytic polypeptides for cleaving target proteins. Each of the recombinant catalytic polypeptide comprises a human antibody light chain that is operably joined to a heterologous heavy chain. The antibody light chain has a serine protease dyad and endopeptidase activity, and the heavy chain has a predetermined activity for the target protein.

In some embodiments, the target proteins are selected from a group consisting of growth factors, cell surface receptors, cytokines, and immunoglobulins. In other embodiments, the human antibody light chain has a serine protease triad. In other embodiments, the recombinant catalytic polypeptide is a single polypeptide chain that contains the human antibody light chain and the antibody heavy chain. In a preferred embodiment, the human antibody light chain comprises an amino acid sequence that has at least 80% identity to SEQ ID NO:2, 4, 6, 8, 10, 12, 14, 16, 18,20, 22,24, 26, or 28. In a more preferred embodiment, the human antibody light chain comprises an amino acid sequence that has at least 95% identity to SEQ ID NO:2, 4, 6, 8, 10, 12, 14, 16, 18, 20, 22, 24, 26, or 28. In a most preferred embodiment, the human antibody light chain comprises an amino acid sequence of SEQ ID NO:2, 4, 6, 8, 10, 12, 14, 16, 18, 20, 22, 24, 26, or 28.

In another aspect, the invention provides for cells hosting a nucleic acid encoding a recombinant catalytic polypeptide for cleaving a target protein. The recombinant catalytic polypeptide comprises a human antibody light chain that is operably joined to a heterologous heavy chain. The antibody light chain has a serine protease dyad and endopeptidase activity, and the heavy chain has a predetermined activity for the target protein.

In some embodiments, the target proteins are selected from a group consisting of growth factors, cell surface receptors, cytokines, and immunoglobulins. In other embodiments, the human antibody light chain has a serine protease triad. In other embodiments, the recombinant catalytic polypeptide is a single polypeptide chain that contains the human antibody light chain and the antibody heavy chain. In a preferred embodiment, the human antibody light chain comprises an amino acid sequence that has at least 80% identity to SEQ ID NO:2, 4, 6, 8, 10, 12, 14, 16, 18, 20, 22, 24, 26, or 28. In a more preferred embodiment, the human antibody light chain comprises an amino acid sequence that has at least 95% identity to SEQ ID NO:2, 4, 6, 8, 10, 12, 14, 16, 18, 20, 22, 24, 26, or 28. In a most preferred embodiment, the human antibody light chain comprises an amino acid sequence of SEQ ID NO:2, 4, 6, 8, 10, 12, 14, 16, 18, 20, 22, 24, 26, or 28.

In another aspect, the invention provides for isolated polypeptides that have a serine protease dyad and endopeptidase activity. Each of the polypeptides comprises an amino acid sequence with at least 80% identity to SEQ ID NO:2, 4, 6, 8, 10, 12, 14, 16, 18, 20, 22, 24, 26, or 28.

In some preferred embodiments, the polypeptides comprise an amino acid sequence with at least 95% identity to SEQ ID NO:2, 4, 6, 8, 10, 12, 14, 16, 18, 20, 22, 24, 26, or 28. In other preferred embodiments, the polypeptides comprise an amino acid sequence of SEQ ID NO:2, 4, 6, 8, 10, 12, 14, 16, 18, 20, 22, 24, 26, or 28. In yet other preferred embodiments, the polypeptides have a serine protease triad.

In another aspect, the invention provides for nucleic acids encoding polypeptides that have a serine protease dyad and endopeptidase activity. Each of the polypeptides comprises an amino acid sequence with at least 80% identity to SEQ ID NO:2, 4, 6, 8, 10, 12, 14, 16, 18, 20, 22, 24, 26, or 28.

In some preferred embodiments, the polypeptides comprise an amino acid sequence with at least 95% identity to SEQ ID NO:2, 4, 6, 8, 10, 12, 14, 16, 18, 20, 22, 24, 26, or 28. In other preferred embodiments, the polypeptides comprise an amino acid sequence of SEQ ID NO:2, 4, 6, 8, 10, 12, 14, 16, 18, 20,22, 24, 26, or 28. In other preferred embodiments, the nucleic acids comprise a nucleic acid sequence of SEQ ID NO: 1, 3, 5, 7, 9, 11, 13, 15, 17, 19, 21, 23, 25, or 27. In yet other preferred embodiments, the polypeptides have a serine protease triad.

In another aspect, the invention provides for cells hosting nucleic acids encoding polypeptides that have a serine protease dyad and endopeptidase activity. Each of the polypeptides comprises an amino acid sequence with at least 80% identity to SEQ ID NO:2, 4, 6, 8, 10, 12, 14, 16, 18, 20, 22, 24, 26, or 28.

In some preferred embodiments, the polypeptides comprise an amino acid sequence with at least 95% identity to SEQ ID NO:2, 4, 6, 8, 10, 12, 14, 16, 18, 20, 22, 24, 26, or 28. In other preferred embodiments, the polypeptides comprise an amino acid sequence of SEQ ID NO:2, 4, 6, 8, 10, 12, 14, 16, 18, 20, 22, 24, 26, or 28. In other preferred embodiments, the nucleic acids comprise a nucleic acid sequence of SEQ ID NO: 1, 3, 5, 7, 9, 11, 13, 15, 17, 19, 21, 23, 25, or 27. In yet other preferred embodiments, the polypeptides have a serine protease triad.

In another aspect, the invention provides for transgenic non-human mammals. The transgene comprises a nucleic acid encoding a polypeptide that has a serine protease dyad and endopeptidase activity. Each of the polypeptides comprises an amino acid sequence with at least 80% identity to SEQ ID NO:2, 4, 6, 8, 10, 12, 14, 16, 18, 20, 22, 24, 26, or 28.

In some preferred embodiments, the polypeptides comprise an amino acid sequence with at least 95% identity to SEQ ID NO:2, 4, 6, 8, 10, 12, 14, 16, 18, 20, 22, 24, 26, or 28. In other preferred embodiments, the polypeptides comprise an amino acid sequence of SEQ ID NO:2, 4, 6, 8, 10, 12, 14, 16, 18, 20, 22, 24, 26, or 28. In other preferred embodiments, the nucleic acids comprise a nucleic acid sequence of SEQ ID NO: 1, 3, 5, 7, 9, 11, 13, 15, 17, 19, 21, 23, 25, or 27. In yet other preferred embodiments, the polypeptides have a serine protease triad.

### BRIEF DESCRIPTION OF THE DRAWINGS

FIG. 1. shows an example of a recombinant catalytic polypeptide embodied in the present invention. Shown are the typical features of a complete antibody molecule, including heavy chain variable region (V_{H}), three heavy chain constant domains (CH1, CH2, CH3), light chain variable region (Vₗ ), and light chain constant region (C_{L}). The complementarity determining regions (CDRs) of the variable domains are also illustrated, including the inclusion of a serine protease dyad in the CDRs of the light chain.

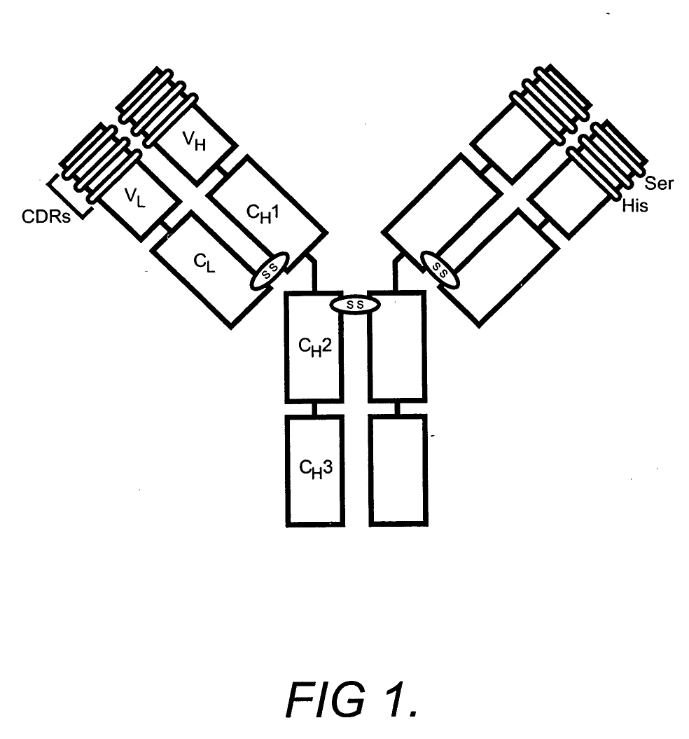

FIG. 2. shows agarose gels of cloned genes encoding the V regions of recombinant catalytic light chains. In the upper panel, primers specific for DNA sequences flanking the A17 V region were used to amplify A17 DNA from human genomic DNA (lane labeled “A17”). In a three-way PCR reaction, the J kappa 1 minigene was fused to A17 (labeled “A17-JK1”). The size of a typical J region is around 50 basepairs. Sizes of the 100 basepair ladder are indicated at the right. The lower panel shows a PCR reaction using primers flanking the A18b V region (labeled “A18b”).

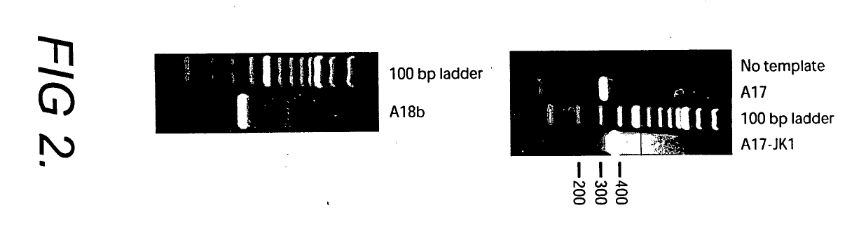

FIG. 3. shows the amino acid sequences (SEQ ID NOS:29-60) of the human kappa light chain repertoire. Those sequences containing serine protease triads are indicated by an asterisk. The aspartate or glutamate component of the triad are underlined and bold, the possible serine components are underlined, and the histidine components are highlighted in black. Position number one is considered a CDR since it is structurally within the antigen combining site.

FIG. 4. shows the purification and activity of germline light chains. LEFT; germline light chains A18b and A2c were purified from the periplasm of E. coli, using two successive columns of nickel resin (ProBond, Invitrogen). The silver stained gel shows the final imidazole elution fractions which included 10, 20, and three 300 mM fractions for each protein. RIGHT; the third 300 mM fraction (400 μl) was dialyzed against 3 L of 20 mM Tris buffer, then incubated with 400 mM of PFR-MCA substrate at 37° C. Fluorescence was quantitated after 24 hrs at 370/465 nm. Asterisks indicate heat deactivation of the protein prior to assay.

FIG. 5. shows identification of proteolytic light chains using a protease triad binding probe. The proteins A18b and A2c, and control factor Xa were incubated with fluorophosphonate probe (middle lane of each group), or heat-denatured prior to incubation with the probe (third lane of each group), run on a 15% SDS-PAGE gel, transferred to a nylon membrane, and incubated with streptavidin conjugated alkaline phosphatase for 1 hour. The membrane was developed with NBT/BCIP reagent.

FIG. 6. shows a phage display vector for proteolytic antibody library generation. The relevant features are shown, including a signal peptide (SP), the invariant light chain with catalytic triad, and CDR positions to be randomized (grey), a flexible linker, library of heavy chains that are fused to gene III of filamentous bacteriophage through a six histidine linker (6× HIS; SEQ ID NO:61). The vector also includes an amber stop codon between the 6× HIS (SEQ ID NO:61) and gene III that allows expression of scFv without fusion to gene III in suppressor E. coli strains. There are convenient restriction sites so that heavy chains or new invariant light chains can be easily inserted into the library.

FIG. 7. shows a Phage ELISA with enrichment of anti-TNF phage through panning. Phage pools obtained through multiple rounds of panning on TNFα were tested for binding to TNFα or interferon-γ (negative control antigen) at 0.5 ug/well. Binding phage were detected by an HRP-conjugated mAb to filamentous phage (fd1) major coat protein (Amersham). The proportion of TNFα binding phage increased with each subsequent pan compared to negative control IFNγ, even as the complexity of each subsequent pan diminished as expected (data not shown).

### DEFINITIONS

A “recombinant catalytic polypeptide” of the present invention comprises an antibody light chain capable of catalyzing hydrolysis of peptide bonds and a heterologous antibody heavy chain. With the two chains operably joined, a recombinant catalytic polypeptide specifically cleaves a target protein.

The term “isolated,” when applied to a nucleic acid or protein, denotes that the nucleic acid or protein is essentially free of other cellular components with which it is associated in the natural state. It is preferably in a homogeneous state although it can be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified. In particular, an isolated gene is separated from open reading frames that flank the gene and encode a protein other than the gene of interest. The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

The term “gene” means the segment of DNA involved in producing a polypeptide chain; it includes regions preceding and following the coding region (leader and trailer) as well as intervening sequences (introns) between individual coding segments (exons).

“Polypeptide” and “peptide” are used interchangeably herein to refer to a polymer of amino acid residues; whereas “protein” may contain one or multiple polypeptide chains. All three terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, y-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid.

Amino acids may be referred to herein by either the commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, “conservatively modified variants” refers to those nucleic acids that encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
(see, e.g., Creighton, Proteins (1984)).

“Percentage of sequence identity” is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, optionally 65%, 70%, 75%, 80%, 85%, 90%, 91%, 92%, 93%, 94%, 95%, 96%, 97%, 98%, 99%, or higher identity over a specified region), when compared and aligned for maximum correspondence over a comparison window, or designated region as measured using one of the following sequence comparison algorithms or by manual alignment and visual inspection. Such sequences are then said to be “substantially identical.” This definition also refers to the complement of a test sequence. Optionally, the identity exists over a region that is at least about 50 nucleotides in length, or more preferably over a region that is 100 to 500 or 1000 or more nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith and Waterman, Adv. Appl. Math. 2:482 (1970), by the homology alignment algorithm of Needleman and Wunsch, J. Mol. Biol. 48:443-453 (1970), by the search for similarity method of Pearson and Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. Nuc. Acids Res. 25:3389-3402 (1977), and Altschul et al. J. Mol. Biol. 215:403-410 (1990), respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=-4 and a comparison of both strands. For amino acid sequences, the BLASTP program uses as defaults a wordlength of 3, and expectation (E) of 10, and the BLOSUM62 scoring matrix (see Henikoff and Henikoff, Proc. Natl. Acad. Sci. USA 89:10915 (1989)) alignments (B) of 50, expectation (E) of 10, M=5, N=-4, and a comparison of both strands.

The BLAST algorithm also performs a statistical analysis of the similarity between two sequences (see, e.g., Karlin and Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The term “cleaving” as used herein refers to the hydrolysis of at least one peptide bond within the amino acid chain of a polypeptide or a protein.

The term “target protein” refers to a polypeptide or protein that is specifically bound and hydrolyzed by a recombinant catalytic polypeptide. Also see the definition of “specificity” below.

An “antibody” refers to a protein of the immunoglobulin family or a polypeptide comprising fragments of an immunoglobulin that is capable of noncovalently, reversibly, and in a specific manner binding a corresponding antigen. An exemplary antibody structural unit comprises a tetramer. Each tetramer is composed of two identical pairs of polypeptide chains, each pair having one “light” (about 25 kD) and one “heavy” chain (about 50-70 kD), connected through a disulfide bond. The recognized immunoglobulin genes include the κ, λ, α, γ, δ, ε, and μ constant region genes, as well as the myriad immunoglobulin variable region genes. Light chains are classified as either κ or λ. Heavy chains are classified as γ, μ, α, δ, or ε, which in turn define the immunoglobulin classes, IgG, IgM, IgA, IgD, and IgE, respectively. The N-terminus of each chain defines a variable region of about 100 to 110 or more amino acids primarily responsible for antigen recognition. The terms variable light chain (Vₗ ) and variable heavy chain (V_{H}) refer to these regions of light and heavy chains respectively.

“Complementarity-determining domains” or “CDRs” refers to the hypervariable regions of V_{L} and V_{H}. The CDRs are the target protein-binding site of the antibody chains that harbors specificity for such target protein. There are three CDRs (CDR1-3, numbered sequentially from the N-terminus) in each human V_{L} or V_{H}, constituting about 15-20% of the variable domains. The CDRs are structurally complementary to the epitope of the target protein and are thus directly responsible for the binding specificity. The remaining stretches of the V_{L} or V_{H}, the so-called framework regions, exhibit less variation in amino acid sequence (Kuby, Immunology, 4th ed., Chapter 4. W.H. Freeman & Co., New York, 2000). Additionally, in the context of the present invention, the first amino acid of V_{H} or V_{L} is considered a CDR since it is structurally within the antigen combining site. Included in this definition of the CDR is any addition of amino acids to the N-terminus of V_{H} or V_{L}.

The positions of the CDRs and framework regions are determined using various well known definitions in the art, e.g., Kabat, Chothia, international ImMunoGeneTics database (IMGT), and AbM (see, e.g., Johnson et al., Nucleic Acids Res., 29:205-206 (2001); Chothia and Lesk, J. Mol. Biol., 196:901-917 (1987); Chothia et al., Nature, 342:877-883 (1989); Chothia et al., J. Mol. Biol., 227:799-817 (1992); Al-Lazikani et al., J. Mol. Biol., 273:927-748 (1997)). Definitions of antigen combining sites are also described in the following: Ruiz et al., Nucleic Acids Res., 28:219-221 (2000); and Lefranc, M. P., Nucleic Acids Res., 29:207-209 (2001); MacCallum et al., J. Mol. Biol., 262:732-745 (1996); and Martin et al, Proc. Natl. Acad. Sci. USA, 86:9268-9272 (1989); Martin et al., Methods Enzymol., 203:121-153 (1991); and Rees et al., In Sternberg M. J. E. (ed.), Protein Structure Prediction, Oxford University Press, Oxford, 141-172 (1996).

An “antibody light chain” or an “antibody heavy chain” as used herein refers to a polypeptide comprising the V_{L} or V_{H}, respectively. The V_{L} is encoded by the gene segments V (variable) and J (junctional), and the V_{H} by V, D (diversity), and J. Each of V_{L} or V_{H} includes the CDRs as well as the framework regions. In this application, antibody light chains and/or antibody heavy chains may, from time to time, be collectively referred to as “antibody chains.” These terms encompass antibody chains containing mutations that do not disrupt the basic structure of V_{L} or V_{H}, as one skilled in the art will readily recognize.

Antibodies exist as intact immunoglobulins or as a number of well-characterized fragments produced by digestion with various peptidases. Thus, for example, pepsin digests an antibody below the disulfide linkages in the hinge region to produce F(_{ab})′₂, a dimer of F_{ab}′ which itself is a light chain joined to V_{H}-C_{H}1 by a disulfide bond. The F(_{ab})!₂ may be reduced under mild conditions to break the disulfide linkage in the hinge region, thereby converting the F(_{ab})′₂ dimer into an F_{ab}′ monomer. The F_{ab}′ monomer is essentially F_{ab} with part of the hinge region. Paul, Fundamental Immunology 3d ed. (1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fᵥ) or those identified using phage display libraries (see, e.g., McCafferty et al., Nature, 348:552-554 (1990))

For preparation of monoclonal or polyclonal antibodies, any technique known in the art can be used (see, e.g., Kohler & Milstein, Nature, 256:495-497 (1975); Kozbor et al., Immunology Today, 4:72 (1983); Cole et al., Monoclonal Antibodies and Cancer Therapy, pp. 77-96. Alan R. Liss, Inc. 1985). Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies, and heteromeric F_{ab} fragments, or scFv fragments that specifically bind to selected antigens (see, e.g., McCafferty et al., supra; Marks et al., Biotechnology, 10:779-783, (1992)).

The term “heterologous” refers to, as used in the context of describing the two antibody chains of the recombinant catalytic polypeptide, the relation between the “antibody light chain” and the “antibody heavy chain” with regard to the sources of their origin. For an “antibody light chain” and an “antibody heavy chain” be “heterologous” to each other, the exact combination of the antibody light and heavy chains must be one that is not found in an antibody produced by a mammal whose genome contains no genetic modification.

The term “endopeptidase activity” as used herein refers to the ability of an enzyme to catalyze the hydrolysis of at least one non-terminal peptide bond between two amino acid residues within a polypeptide of any length.

The “specificity” for a target protein refers to the ability of antibody heavy chain of a recombinant catalytic polypeptide to distinguish between the target protein and any other polypeptides, based on their structural difference, such that the binding between the target protein and the antibody heavy chain under designated conditions is to a reasonable degree unique. For example, the binding between an antibody and a target protein is deemed specific when a signal at least two times over background is detected in a binding assay. A “predetermined specificity” for a target protein is achieved by either isolating the heavy chain of a known antibody against a pre-selected target protein, or screening a repertoire of in vivo generated antibody gene products for specific binding to that particular target protein. These heavy chains may also be further modified for enhanced specificity.

Despite the diversity in primary amino acid sequence among individual members of the family, serine protease activity is supported by a highly conserved tertiary structure, which comprises a serine-histidine-aspartate triad. Studies have shown that the aspartate residue is not always essential for catalytic activity. The “serine protease dyad” as used herein is the minimal structure of the catalytic site for a recombinant catalytic polypeptide to maintain at least a portion of its proteolytic activity. This structure comprises a histidine residue and a serine residue located within any CDR of an antibody light chain, where the residues are in a spatial relation to each other similar to their spatial alignment in a serine protease triad, such that the histidine can abstract the proton from the serine hydroxyl group, allowing the serine to act as a nucleophile and attack the carbonyl group of the amide bond within the protein substrate.

The “enzymatic activity” of a recombinant catalytic polypeptide as used herein refers to two separate aspects of the polypeptide's characteristics: first, the polypeptide's ability to bind to a target protein with specificity under designated conditions; second, the polypeptide's ability to hydrolyze at least one non-terminal peptide bond within the target protein.

The two heterologous human antibody chains are “operably joined” when they are placed in a functional relationship with each other, such that the manner in which they are joined allows each chain to function properly in binding the target protein with specificity and catalyzing the hydrolysis of the target protein. The methods of operably joining two antibody chains include but are not limited to, recombinant fusion by a linker peptide, covalent bonding, disulfide bonding, ionic bonding, hydrogen bonding, and electrostatic bonding.

“Mutating” or “mutation” as used in the context of altering the enzymatic activity of a recombinant catalytic polypeptide refers to the deletion, insertion, or substitution of any nucleotide, by chemical, enzymatic, or any other means, in a nucleic acid encoding a recombinant catalytic polypeptide such that the amino acid sequence of the resulting polypeptide is altered at one or more amino acid residues.

A “library” of recombinant catalytic polypeptide members refers to a repertoire of recombinant polypeptides that are capable of catalyzing the hydrolysis of non-terminal peptide bonds within a polypeptide. The recombinant polypeptide library comprises members with distinct substrate specificities, determined by the CDRs of the member's antibody heavy chain.

The phrase “a nucleic acid sequence encoding a recombinant catalytic polypeptide” refers to a nucleic acid which contains sequence information for the amino acid sequence of a recombinant catalytic polypeptide. This phrase specifically encompasses degenerate codons (i.e., different codons which encode a single amino acid) of the native sequence or sequences which may be introduced to conform with codon preference in a specific host cell. As used in this phrase, a “nucleic acid” refers to deoxyribonucleotides or ribonucleotides and polymers thereof in either single- or double-stranded form. The term encompasses nucleic acids containing known nucleotide analogs or modified backbone residues or linkages, which are synthetic or naturally-occurring, having similar binding properties as the reference nucleic acid, and metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

The term “growth factor” as used herein refers to any peptide that is capable of inducing proliferation of any particular cell type under designated conditions. The term encompasses all polypeptides encoded by wild-type genes and genes with mutations.

“Cytokine” refers to small, biologically active polypeptides produced by a variety of cells. These polypeptides generally act as intercellular mediators, with multiple potential targets as well as multiple potential functions, such as to signal cell proliferation, differentiation, or apoptosis. Examples of cytokines include lymphokines, interleukins, interferons, etc. “Cytokine” as used here encompasses all polypeptides encoded by wild-type genes and genes with mutations.

“The EGFR family” as used herein refers to the four members of the epidermal growth factor receptor family, EGFR, HER2/neu, ErbB-3, and ErbB-4. The term encompasses all polypeptides encoded by wild-type genes and genes with mutations.

### DETAILED DESCRIPTION OF THE INVENTION

I. Introduction

Many proteins have been identified to play important roles in the pathogenesis and progression of a variety of human diseases and conditions. For example, vascular endothelial growth factor (VEGF) has been shown in clinical and experimental studies to promote angiogenesis, a prerequisite for solid tumor growth (see, e.g., Plate et al., Nature, 359:845-848 (1992); Smith, Hum. Reprod. Update, 4:509-519 (1998)). Another example is the four members of the epidermal growth factor receptor family, EGFR, HER2/neu, ErbB-3, and ErbB-4, which are involved in cell proliferation, differentiation, and survival. In particular, the overexpression of EGFR and HER2/neu is frequently found in, e.g., lung cancers and breast cancers, respectively (see, e.g., Franklin et al., Semin. Oncol., 29:3-14 (2002)). A third example is IgE, the hyperproduction of which has long been associated with a number of immunological disorders such as asthma (see, e.g., Romagnani, Immunol. Today, 11:316-321 (1990)). Though the reduction of the level of these proteins is thought to be critical for treating these diseases and conditions, naturally-occurring proteases cannot be used as a means of treatment since there are no known proteases that can specifically hydrolyze these proteins. Numerous therapeutic approaches targeting these proteins have been developed, such as using inhibitory agents or antisense nucleotide sequences to suppress their expression, or using antibodies to neutralized their functions (see, e.g., U.S. Pat. Nos. 5,760,041, 6,150,092, and 6,416,758; Babu and Holgate, Indian J. Chest Dis. Allied Sci., 44:107-115 (2002)). It is not unusual, however, to observe a varying degree of effectiveness when these general methods are used in therapy, a phenomenon in part due to insufficient level of specificity of these therapeutic agents for the target proteins.

The present invention provides an innovative solution to this problem, utilizing a mechanism previously associated with only the human immune system, which is capable of generating antibodies with high level of specificity for virtually any antigen. By operably joining two heterologous human antibody chains, one of which supplies the catalytic activity to hydrolyze polypeptides and the other the binding specificity for a target protein, the present invention teaches the construction of a repertoire of proteases with customized protein substrate specificities of potentially unlimited number and thus makes possible the effective treatment and/or prevention of any medical condition attributable to the presence or overexpression of an identified protein.

II. Construction of Antibody Chains of the Recombinant Catalytic Polypeptides

A. Obtaining Nucleic Acid Sequences

(1) Overview

This invention relies on routine techniques in the field of recombinant genetics. Basic texts disclosing the general methods of use in this invention include Sambrook and Russell, Molecular Cloning: A Laboratory Manual 3d ed. (2001); Kriegler, Gene Transfer and Expression: A Laboratory Manual (1990); and Ausubel et al., Current Protocols in Molecular Biology (1994).

For nucleic acids, sizes are given in either kilobases (Kb) or base pairs (bp). These are estimates derived from agarose or polyacrylamide gel electrophoresis, from sequenced nucleic acids, or from published DNA sequences. For proteins, sizes are given in kilo-Daltons (kD) or amino acid residue numbers. Proteins sizes are estimated from gel electrophoresis, from sequenced proteins, from derived amino acid sequences, or from published protein sequences.

Oligonucleotides that are not commercially available can be chemically synthesized according to the solid phase phosphoramidite triester method first described by Beaucage and Caruthers, Tetrahedron Letters, 22:1859-1862 (1981), using an automated synthesizer, as described in Van Devanter et al., Nucleic Acids Res., 12:6159-6168 (1984). Purification of oligonucleotides is by either native polyacrylamide gel electrophoresis or by anion-exchange chromatography as described in Pearson & Reanier, J. Chrom., 255:137-149 (1983). The sequence of the cloned genes and synthetic oligonucleotides can be verified after cloning using, e.g., the chain termination method for sequencing double-stranded templates of Wallace et al., Gene, 16:21-26 (1981).

(2) Nucleotide Sequences Encoding Antibody Light Chains with Proteolytic Activity

In general, a nucleic acid sequence encoding the V region of an antibody light chain with proteolytic activity of human origin, e.g., SEQ ID NO:1, 3, 5, 7, 9, 11, 13, 15, 17, 19, 21, 23, 25, or 27, is obtained based on an expected sequence homology to a nucleic acid encoding a proteolytic antibody light chain V region that has already been cloned from another species. Genes encoding the constant regions for human κ and λ light chains are known and can be subsequently fused to genes encoding V_{L} with desired proteolytic activity (e.g., SEQ ID NO:1, 3, 5, 7, 9, 11, 13, 15, 17, 19, 21, 23, 25, or 27) to generate coding sequences for full length antibody light chains.

The rapid progress in the studies of human genome has made possible a cloning approach where a human DNA sequence database can be searched for any gene segment that has a certain percentage of sequence homology to a known nucleotide sequence, such as one encoding a murine proteolytic antibody light chain. Any DNA sequence so identified can be subsequently obtained by chemical synthesis and/or polymerase chain reaction (PCR) such as overlap extension method. For a short sequence, completely de novo synthesis may be sufficient; whereas further isolation of full length coding sequence from a human cDNA or genomic library using a synthetic probe may be necessary to obtain a larger gene. Most commonly used techniques for such purpose are described in, e.g., Sambrook and Russell, supra and White et al., supra.

Alternatively, a nucleic acid sequence encoding an antibody light chain V region with proteolytic activity such as SEQ ID NO:1, 3, 5, 7, 9, 11, 13, 15, 17,19, 21, 23, 25, or 27 can be isolated from human cDNA or genomic DNA libraries using standard cloning techniques such as PCR. Primers can be derived from a known nucleic acid sequence encoding an antibody light chain V region with proteolytic activity in another species, e.g., a murine proteolytic light chain V region sequence.

Human cDNA libraries suitable for obtaining coding sequence for a proteolytic antibody light chain V region may be commercially available or can be constructed. Since proteolytic antibodies often can be found in patients suffering from various autoimmune diseases (see, e.g., Paul et al., Science, 244:1158-1162 (1989); Thiagarajan et al., Biochemistry, 39:6459-6465 (2000)), such a cDNA library can be constructed using a source likely to contain high level of mRNA encoding proteolytic autoantibodies, such as B cells from a patient with autoimmune disease. The general methods of isolating mRNA, making cDNA by reverse transcription, ligating cDNA into a recombinant vector, and transfecting into a recombinant host for propagation, screening, and cloning are well known (see, e.g., Gubler and Hoffmnan, Gene, 25:263-269 (1983); Ausubel et al., supra). Upon obtaining an amplified segment of nucleotide sequence by PCR, the segment can be further used as a probe to isolate the full length nucleic acid encoding the antibody chain with proteolytic activity from the cDNA library. General description of the procedure can be found in Sambrook and Russell, supra.

A similar procedure can be followed to obtain a full length sequence encoding a proteolytic antibody light chain V region, e.g., SEQ ID NO:1, 3, 5, 7, 9, 11, 13, 15, 17, 1,9, 21, 23, 25, or 27, from a human genomic library. Human genomic libraries may be commercially available or can be constructed according to methods described in scientific literature. In general, to construct a genomic library, the DNA is first extracted from an organism where the proteolytic antibodies are likely found, and either mechanically sheared or enzymatically digested to yield fragments of about 12-20 kb in length. The fragments are then separated by gradient centrifugation from undesired sizes and are constructed in bacteriophage λ vectors. These vectors and phages are packaged in vitro. Recombinant phages are analyzed by plaque hybridization as described in Benton and Davis, Science, 196:180-182 (1977). Colony hybridization is carried out as described by Grunstein et al., Pro. Natl. Acad. Sci. USA, 72:3961-3965 (1975).

Based on sequence homology, degenerate oligonucleotides can be designed as primer sets and PCR can be performed under suitable conditions (see, e.g., White et al., PCR Protocols: Current Methods and Applications, 1993; Griffin and Griffin, PCR Technology, CRC Press Inc. 1994) to amplify a segment of nucleotide sequence from a human cDNA or genomic library. Using the segment as a probe, full length nucleic acid encoding the entire proteolytic antibody light chain can be obtained subsequently.

Upon acquiring nucleic acid sequence encoding a proteolytic antibody light chain V region, e.g., SEQ ID NO:1, 3, 5, 7, 9, 11, 13, 15, 17, 19, 21, 23, 25, or 27, further modifications to the sequence can be made to provide diversity in various properties, particularly enzymatic activity, of the recombinant polypeptide. One skilled in the art will know many such methods for creating variants, which are described in detail in a later section.

From an encoding nucleic acid sequence, the amino acid sequence of a proteolytic antibody light chain V region, e.g., SEQ ID NO:2, 4, 6, 8, 10, 12, 14, 16, 18, 20, 22, 24, 26, or 28, can be deduced and the presence of a serine protease dyad can be confirmed. The amino acid sequence for a full length proteolytic antibody light chain can be similarly determined. A serine protease dyad comprises serine and histidine residues, which are required for catalytic activity. Such catalytic activity can be detected in an assay described in a later section. Functionally, a serine protease dyad may be identified by site-directed mutagenesis. In other words, catalytic activity of the light chain should be nearly completely abolished when either of the two residues is substituted by another amino acid. Structurally, a serine protease dyad can be identified by, e.g., X-ray crystallography and computer-based programs. The serine and histidine residues in the dyad will be readily identifiable for their three-dimensional juxtaposition in the crystal structure of the antibody light chain. Furthermore, crystal structure of the antibody light chain/transition-state substrate complex will allow identification of the residues by virtue of their proximity to the scissile bond of the substrate. The spatial alignment of amino acid residues of a catalytic antibody light chain can also be generated using computer-based methods, such as molecular modeling, known to those skilled in the art and superimposed on the highly conserved tertiary structure of the catalytic site of a known serine protease (such as subtilisin), a serine protease dyad can be subsequently identified (see description of methods and device in, e.g., Gao et al., J. Bio. Chem., 269:32389-32393 (1994)).

(3) Nucleotide Sequences Encoding Antibody Heavy Chains with Specificity for Target Proteins

i. Cloning Nucleotide Sequences

In the construction of recombinant catalytic polypeptides of the instant invention, antibody heavy chains, which provide specificity to bind particular target proteins, can be selected from naturally-occurring antibodies with known specificity for target proteins. In particular, the most preferable antibody heavy chains are from antibodies the antigen specificity of which primarily depends on the heavy chains rather than the light chains. Various assays are known to those skilled in the art to separate an antibody heavy chain from an antibody light chain, and determine whether the heavy chain has higher affinity to an antigen, i.e., whether it is predominantly responsible for antigen specificity (see, e.g., Edelman et al, Pro. Natl. Acad. Sci. USA, 50:753-761 (1963); Utsumi et al, Biochemistry, 9:1329-1342 (1964); Sun et al, J. Biol. Chem., 269:734-738 (1994)).

In some cases, nucleotide sequence encoding a suitable antibody heavy chain may already have been determined in previous studies and the sequence can be used directly in producing the recombinant catalytic polypeptide of the instant invention. For the antibody heavy chains whose encoding sequences have not been previously cloned, the same general cloning methods as described above are also suitable for isolating genes encoding antibody heavy chains. The heavy chain of an antibody against a particular antigen can be isolated using affinity chromatography and electrophoresis. Its partial amino acid sequence can then be determined and full length nucleotide sequence can be isolated from a cDNA library or a genomic DNA library, relying on standard cloning techniques. The nucleotide sequence can also be obtained based on sequence homology of the antibody of interest in another species.

ii. In vitro Generation of Antibody Heavy Chain Genes

An alternative means of obtaining nucleic acids encoding antibody heavy chains for recombinant catalytic polypeptides of the present invention is via in vitro recombination of gene segments. This method generates more target protein specificities and is especially useful when no naturally-occurring antibodies can provide suitable heavy chains with desired specificity against particular target proteins.

The constant region of a heavy chain is encoded by a constant region gene (C), whereas the genomic structure of the variable region of a heavy chain is composed of three gene segment families. These segments are termed variable (V), diversity (D), and junctional (J). Antibody heavy chain genes produce an array of diversity to allow a variable region repertoire to bind virtually any three dimensional antigenic structure. Three distinct genetic mechanisms are used in generating such diversity: (1) V(D)J recombination between gene segments; (2) junctional diversity created at the V-D, D-J, or V-J junctional sequences; and (3) somatic hypermutation.

Diversity in heavy chain variable region can also be generated in vitro by coupling antibody gene segments, which may be of the V, D, J, or C varieties. The gene segments can be of germline sequence, or can be sequences related to germline sequence. The gene segments may be from any organism and the gene segments from different organisms may be coupled to one another in any order. The coupling reaction produces at least one phosphodiester bond linking at least two gene segments together, by chemical, enzymatic, or any other means. A number of well established techniques that can be used in recombining the gene segments includes, for example, ligation of nucleic acid and/or PCR assembly following DNase digestion, and synthetic recombination methods.

The coupling of gene segments may occur with the loss or gain of nucleotides at the coupled joint. Such a loss or gain of residues adds diversity at the amino acid residues that contact antigen, and can provide improved antibody function. The loss of nucleotides at the joint can be accomplished by enzymatic means, e.g., using exonuclease to remove nucleotides from the ends of the gene segments. Methods of creating deletions at the end of a nucleic acid using exonuclease III are described in patent application PCT/US 01/25788. Nucleotides may also be added by enzymatic means, such as using terminal deoxynucleotidyl transferase to add nucleotides to the 3′ end of a gene segment. Alternatively, nucleotides may be added or removed by chemical means. For example, nucleotides can be added to the end of a gene segment during oligonucleotide synthesis, to the end of a PCR primer used to amplify a particular gene segment, or internally in a PCR primer capable of hybridizing simultaneously to the two segments to be coupled. Similarly, nucleotides may also be deleted by not incorporating the terminal nucleotides during gene synthesis or by synthesis shortened primers for PCR amplification of gene segments. The methods of generating antibody heavy chain genes with enhanced diversity are disclosed in patent application No. 60/337,718, which is incorporated herein in its entirety by reference.

The newly formed antibody gene segments can be further diversified by various procedures, which are analogous to the in vivo mechanism of somatic hypermutation. The description for these procedures is provided in the following section.

B. Modifications of Nucleotide Sequences for Diversity

In order to achieve enhanced enzymatic activity and more diverse target protein specificity, further modifications can be made to nucleotide sequences encoding antibody chains of recombinant catalytic polypeptides of the invention, whether such sequences are naturally-occurring or generated in vitro.

A variety of diversity-generating protocols have been established and described in the art. See, e.g., Zhang et al., Proc. Natl. Acad. Sci. USA, 94:4504-4509 (1997); and Stemmer, Nature, 370:389-391 (1994). The procedures can be used separately or in combination to produce variants of a set of nucleic acids, and hence variants of encoded polypeptides. Kits for mutagenesis, library construction, and other diversity-generating methods are commercially available.

Mutational methods of generating diversity include, for example, site-directed mutagenesis (Botstein and Shortle, Science, 229:1193-1201 (1985)), mutagenesis using uracil-containing templates (Kunkel, Proc. Natl. Acad. Sci. USA, 82:488-492 (1985)), oligonucleotide-directed mutagenesis (Zoller and Smith, Nucl. Acids Res., 10:6487-6500 (1982)), phosphorothioate-modified DNA mutagenesis (Taylor et al., Nucl. Acids Res., 13:8749-8764 and 8765-8787 (1985)), and mutagenesis using gapped duplex DNA (Kramer et al., Nucl. Acids Res., 12:9441-9456 (1984)).

Other suitable methods include point mismatch repair (Kramer et al., Cell, 38:879-887 (1984)), mutagenesis using repair-deficient host strains (Carter et al., Nucl. Acids Res., 13:4431-4443 (1985)), deletion mutagenesis (Eghtedarzadeh and Henikoff, Nucl. Acids Res., 14:5115 (1986)), restriction-selection and restriction-purification (Wells et al., Phil. Trans. R. Soc. Lond. A, 317:415-423 (1986)), mutagenesis by total gene synthesis (Nambiar et al., Science, 223:1299-1301 (1984)), double-strand break repair (Mandecki, Proc. Natl. Acad. Sci. USA, 83:7177-7181 (1986)), mutagenesis by polynucleotide chain termination methods (U.S. Pat. No. 5,965,408), and error-prone PCR (Leung et al., Biotechniques, 1:11-15 (1989)).

Diversity also can be generated in nucleic acids or populations of nucleic acids using a recombinational procedure termed “incremental truncation for the creation of hybrid enzymes” (“ITCHY”) described in Ostermeier et al., Nature Biotech., 17:1205 (1999). This approach can be used to generate an initial library of variants which can optionally serve as a substrate for one or more in vitro or in vivo recombination methods. See, also, Ostermeier et al., Proc. Natl. Acad. Sci. USA, 96:3562-67 (1999); Ostermeier et al., Bio. Me. Chem., 7:2139-44 (1999).

By using the methods described above, a large number of nucleic acid variants can be derived from wild type sequences or in vitro generated sequences encoding antibody chains of the recombinant catalytic polypeptides. Since not all diversity is functional, the recombinant polypeptide variants should be screened for their ability to bind and hydrolyze target proteins in assays described in a later section.

Alternatively, it may be desirable to pre-select or bias the substrates towards nucleic acids that encode functional products prior to diversification. In the case of antibody heavy chain engineering, for instance, it is possible to bias the diversity generating process toward heavy chains with functional antigen binding sites by taking advantage of in vivo recombination events prior to manipulation by any of the described methods. One such example is to amplify recombined CDRs derived from B cell cDNA libraries and then assemble them into the framework regions (see, e.g., Jirholt et al., Gene, 215:471-476 (1998)) prior to diversification. Nucleic acid libraries can also be biased towards nucleic acids encoding polypeptides with desirable enzyme activities (see, e.g., U.S. Pat. No. 5,939,250).

C. Modifications of Nucleic Acids for Preferred Codon Usage in an Organism

The polynucleotide sequence encoding a particular recombinant catalytic polypeptide can be altered to coincide with the preferred codon usage of a particular host. For example, the preferred codon usage of one strain of bacteria can be used to derive a polynucleotide that encodes a recombinant catalytic polypeptide of the invention and comprises the codons favored by this strain. The frequency of preferred codon usage exhibited by a host cell can be calculated by averaging frequency of preferred codon usage in a large number of genes expressed by the host cell (see for example, http://www.kazusa.orjp/codon/). This analysis is preferably limited to genes that are highly expressed by the host cell. U.S. Pat. No. 5,824,864, for example, provides the frequency of codon usage by highly expressed genes exhibited by dicotyledonous plants and monocotyledonous plants.

III. Expression in Prokaryotes and Eukaryotes

A. Cells for Expression of Recombinant Polypeptides

Various cell types, both prokaryotic and eukaryotic, are suitable for the expression of the recombinant catalytic polypeptides or the proteolytic antibody light chains (e.g., polypeptides comprising an amino acid sequence of SEQ ID NO:2, 4, 6, 8, 10, 12, 14, 16, 18, 20, 22, 24, 26, or 28) of the present invention. These cell types include but are not limited to, for example, a variety of bacteria such as E. coli, Bacillus sp., and Salmonella, as well as eukaryotic cells such as yeast, insect cells, and mammalian cells. Suitable cells for gene expression are well known to those of skill in the art and are described in numerous scientific publications such as Sambrook and Russell, supra.

B. Expression Vectors

The nucleic acids encoding recombinant polypeptides of the present invention are typically cloned into an intermediate vector before transformation into prokaryotic or eukaryotic cells for replication and/or expression. The intermediate vector is typically a prokaryote vector such as a plasmid or shuttle vector.

To obtain high level expression of a cloned gene, such as the cDNA encoding aproteolytic antibody chain comprising SEQ ID NO:1, 3, 5, 7, 9, 11, 13, 15, 17, 19,21,23, 25, or 27, one typically subdlones the cDNA into an expression vector that contains a strong promoter to direct transcription, a transcription/translation terminator, and a ribosome binding site for translational initiation. Suitable bacterial promoters are well known in the art and fully described in scientific literature such as Sambrook and Russell, supra, and Ausubel et al, supra. Bacterial expression systems for expressing antibody chains of the recombinant e catalytic polypeptide are available in, e.g., E. coli, Bacillus sp., and Salmonella (Palva et al., Gene, 22:229-235 (1983); Mosbach et al., Nature, 302:543-545 (1983)). Kits for such expression systems are commercially available. Eukaryotic expression systems for mammalian cells, yeast, and insect cells are well known in the art and are also commercially available.

Selection of the promoter used to direct expression of a heterologous nucleic acid depends on the particular application. The promoter is preferably positioned about the same distance from the heterologous transcription start site as it is from the transcription start site in its natural setting. As is known in the art, however, some variation in this distance can be accommodated without loss of promoter function.

In addition to the promoter, the expression vector typically contains a transcription unit or expression cassette that contains all the additional elements required for the expression of the proteolytic antibody chain in host cells. A typical expression cassette thus contains a promoter operably linked to the nucleic acid sequence encoding the proteolytic antibody chain and signals required for efficient polyadenylation of the transcript, ribosome binding sites, and translation termination. Additional elements of the cassette may include enhancers and, if genomic DNA is used as the structural gene, introns with functional splice donor and acceptor sites.

In addition to a promoter sequence, the expression cassette should also contain a transcription termination region downstream of the structural gene to provide for efficient termination. The termination region may be obtained from the same gene as the promoter sequence or may be obtained from different genes.

The particular expression vector used to transport the genetic information into the cell is not particularly critical. Any of the conventional vectors used for expression in eukaryotic or prokaryotic cells may be used. Standard bacterial expression vectors include plasmids such as pBR322 based plasmids, pSKF, pET23D, and fusion expression systems such as MBP, GST, and LacZ. Epitope tags can also be added to recombinant proteins to provide convenient methods of isolation, e.g., c-myc or histidine tags.

Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A⁺, pMTO1 0/A⁺, pMAMneo-5, baculovirus pDSVE, and any other vector allowing expression of proteins under the direction of the CMV promoter, SV40 early promoter, SV40 later promoter, metallothionein promoter, murine mammary tumor virus promoter, Rous sarcoma virus promoter, polyhedrin promoter, or other promoters shown effective for expression in eukaryotic cells.

Some expression systems have markers that provide gene amplification such as thymidine kinase and dihydrofolate reductase. Alternatively, high yield expression systems not involving gene amplification are also suitable, such as using a baculovirus vector in insect cells, with a nucleic acid sequence encoding a proteolytic antibody chain under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. The prokaryotic sequences are preferably chosen such that they do not interfere with the replication of the DNA in eukaryotic cells, if necessary.

C. Transfection Methods

Standard transfection methods are used to produce bacterial, mammalian, yeast, or insect cell lines that express large quantity of antibody chains of the recombinant catalytic polypeptide, which is then purified using standard techniques (see, e.g., Colley et al., J. Biol. Chem., 264:17619-17622 (1989); Guide to Protein Purification, in Methods in Enzymology, vol. 182 (Deutscher, ed.), 1990). Transformation of eukaryotic and prokaryotic cells are performed according to standard techniques (see, e.g., Morrison, J. Bact., 132:349-351 (1977); Clark-Curtiss and Curtiss, Methods in Enzymology, 101:347-362 (Wu et al., eds), (1983)).

Any of the well-known procedures for introducing foreign nucleotide sequences into host cells may be used. These include the use of calcium phosphate transfection, polybrene, protoplast fusion, electroporation, biolistics, liposomes, microinjection, plasma vectors, viral vectors and any of the other well known methods for introducing cloned genomic DNA, cDNA, synthetic DNA, or other foreign genetic material into a host cell (see, e.g., Sambrook and Russell, supra). It is only necessary that the particular genetic engineering procedure used be capable of successfully introducing at least both genes into the host cell capable of expressing the recombinant catalytic polypeptide.

After the expression vector is introduced into the cells, the transfected cells are cultured under conditions favoring expression of the proteolytic antibody chain, which is recovered from the culture using standard techniques identified below.

D. Screening for Cells Expressing Recombinant Polypeptide

Following the transfection procedure, cells are screened for the expression of antibody chains of the recombinant catalytic polypeptide.

Several general methods for screening gene expression are well known among those skilled in the art. First, gene expression can be detected at nucleic acid level. A variety of methods of specific DNA and RNA measurement using nucleic acid hybridization techniques are commonly used (e.g., Sambrook and Russell, supra). Some methods involve an electrophoretic separation (e.g., Southern blot for detecting DNA and Northern blot for detecting RNA), but detection of DNA or RNA can be carried out without electrophoresis as well (such as by dot blot). The presence of nucleic acid encoding recombinant catalytic polypeptide in transfected cells can also be detected by PCR or RT-PCR using sequence-specific primers.

Second, gene expression can be detected at the polypeptide level. Various immunological assays are routinely used by those skilled in the art to measure the level of a gene product, particularly using polyclonal or monoclonal antibodies that react specifically with a recombinant polypeptide of the present invention, such as an antibody light chain comprising the amino acid sequence of SEQ ID NO:2, 4, 6, 8, 10, 12, 14, 16, 18, 20, 22, 24, 26, or 28, (e.g., Harlow and Lane, Antibodies, A Laboratory Manual, Chapter 14, Cold Spring Harbor, 1988; Kohler and Milstein, Nature, 256:495-497 (1975)). Such techniques require antibody preparation by selecting antibodies with high specificity against the recombinant polypeptide or an antigenic portion thereof. The methods of raising polyclonal and monoclonal antibodies are well established and their descriptions can be found in the literature, see, e.g., Harlow and Lane, supra; Kohler and Milstein, Eur. J. Immunol., 6:511-519 (1976).

In addition, functional assays may also be performed for the detection of recombinant catalytic polypeptide or proteolytic light chain comprising an amino acid sequence of, e.g., SEQ ID NO:2, 4, 6, 8, 10, 12, 14, 16, 18, 20, 22, 24, 26, or 28, in transfected cells. Assays for detecting binding specificity for a predetermined target protein and assays for proteolytic activity of the recombinant catalytic polypeptide are generally described in a later section.

IV. Purification of the Recombinant Polypeptides

Either naturally-occurring or recombinant antibody chains of the recombinant catalytic polypeptides of the present invention can be purified for use in functional assays. Naturally-occurring proteolytic antibody light chains can be purified, for example, from the B cells or serum of a human patient who has been identified to produce proteolytic autoantibodies. Recombinant antibody chains such as antibody light chains comprising an amino acid sequence of SEQ ID NO:2, 4, 6, 8, 10, 12, 14, 16,;18, 20, 22, 24, 26, or 28 can be purified from any suitable expression system discussed above.

The recombinant catalytic polypeptides of the invention may be purified to substantial purity by standard techniques, including selective precipitation with such substances as ammonium sulfate; column chromatography, gel filtration, immunopurification methods, and others (see, e.g., U.S. Pat. No. 4,673,641; Scopes, Protein Purification: Principles and Practice, 1982; Sambrook and Russell, supra; and Ausubel et al., supra).

A number of procedures can be employed when recombinant catalytic polypeptides are purified. For example, proteins having established molecular adhesion properties can be reversibly fused to polypeptides of the invention. With the appropriate ligand, the polypeptides can be selectively adsorbed to a purification column and then freed from the column in a relatively pure form. The fused protein is then removed by enzymatic cleavage. Finally the polypeptide can be purified using affinity columns.

A. Purification of Recombinant Polypeptides from Bacteria

When recombinant polypeptides are expressed by the transformed bacteria in large amounts, typically after promoter induction, although expression can be constitutive, the polypeptides may form insoluble aggregates. There are several protocols that are suitable for purification of polypeptide inclusion bodies. For example, purification of aggregate polypeptides (hereinafter referred to as inclusion bodies) typically involves the extraction, separation and/or purification of inclusion bodies by disruption of bacterial cells typically, but not limited to, by incubation in a buffer of about 100-150 μg/ml lysozyme and 0. 1% Nonidet P40, a non-ionic detergent. The cell suspension can be ground using a Polytron grinder (Brinkman Instruments, Westbury, N.Y.). Alternatively, the cells can be sonicated on ice. Additional methods of lysing bacteria are described in detail in numerous scientific publications (such as Sambrook and Russell, supra, and Ausubel et al., supra), and will be apparent to those of skill in the art.

The cell suspension is generally centrifuged and the pellet containing the inclusion bodies resuspended in buffer which does not dissolve but washes the inclusion bodies, e.g., 20 mM Tris-HCl (pH 7.2), 1 mM EDTA, 150 mM NaCl and 2% Triton-X 100, a non-ionic detergent. It may be necessary to repeat the wash step to remove as much cellular debris as possible. The remaining pellet of inclusion bodies may be resuspended in an appropriate buffer (e.g., 20 mM sodium phosphate, pH 6.8, 150 mM NaCl). Other appropriate buffers will be apparent to those of skill in the art.

Following the wash step, the inclusion bodies are solubilized by the addition of a solvent that is both a strong hydrogen acceptor and a strong hydrogen donor (or a combination of solvents each having one of these properties). The recombinant polypeptides that formed the inclusion bodies may then be renatured by dilution or dialysis with a compatible buffer. Suitable solvents include, but are not limited to, urea (from about 4 M to about 8 M), formamide (at least about 80%, volume/volume basis), and guanidine hydrochloride (from about 4 M to about 8 M). Some solvents that are capable of solubilizing aggregate-forming polypeptides, such as sodium dodecyl sulfate (SDS) and 70% formic acid, are inappropriate for use in this procedure due to the possibility of irreversible denaturation of the polypeptides, accompanied by a lack of binding specificity and/or catalytic activity. Although guanidine hydrochloride and similar agents are denaturants, this denaturation is not irreversible and renaturation may occur upon removal (by dialysis, for example) or dilution of the denaturant, allowing re-formation of the biologically active recombinant catalytic polypeptides. After solubilization, the polypeptides can be separated from other bacterial proteins by standard separation techniques.

Alternatively, it is possible to purify recombinant catalytic polypeptides or proteolytic antibody light chains (e.g., those comprising the amino acid sequence of SEQ ID NO:2, 4, 6, 8, 10, 12, 14, 16, 18, 20, 22, 24, 26, or 28) from bacteria periplasm. Where the polypeptide is exported into the periplasm of the bacteria, the periplasmic fraction of the bacteria can be isolated by cold osmotic shock in addition to other methods known to those of skill in the art (e.g., Ausubel et al., supra). To isolate recombinant polypeptides from the periplasm, the bacterial cells are centrifuged to form a pellet. The pellet is resuspended in a buffer containing 20% sucrose. To lyse the cells, the bacteria are centrifuged and the pellet is resuspended in ice-cold 5 mM MgSO₄ and kept in an ice bath for approximately 10 minutes. The cell suspension is centrifuged and the supernatant decanted and saved. The recombinant polypeptides present in the supernatant can be separated from the host proteins by standard separation techniques well known to those of skill in the art.

B. Standard Protein Separation Techniques for Purifying Proteins

(1) Solubility Fractionation

Often as an initial step, and if the protein mixture is complex, an initial salt fractionation can separate many of the unwanted host cell proteins (or proteins derived from the cell culture media) from the recombinant polypeptides of the invention. The preferred salt is ammonium sulfate. Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol is to add saturated ammonium sulfate to a protein solution so that the resultant ammonium sulfate concentration is between 20-30%. This will precipitate the most hydrophobic proteins. The precipitate is discarded (unless the recombinant catalytic polypeptide is hydrophobic) and ammonium sulfate is added to the supernatant to a concentration known to precipitate the recombinant polypeptide. The precipitate is then solubilized in buffer and the excess salt removed if necessary, through either dialysis or diafiltration. Other methods that rely on solubility of proteins, such as cold ethanol precipitation, are well known to those of skill in the art and can be used to fractionate complex protein mixtures.

(2) Size Differential Filtration

Based on a calculated molecular weight, a polypeptide of greater and lesser size can be isolated using ultrafiltration through membranes of different pore sizes (for example, Amicon or Millipore membranes). As a first step, the protein mixture is ultrafiltered through a membrane with a pore size that has a lower molecular weight cut-off than the molecular weight of the recombinant catalytic polypeptide or the proteolytic antibody light chain. The retentate of the ultrafiltration is then ultrafiltered against a membrane with a molecular cut-off greater than the molecular weight of the recombinant catalytic polypeptide or the proteolytic antibody light chain. The polypeptide will pass through the membrane into the filtrate. The filtrate can then be processed in a next step of column chromatography.

(3) Column Chromatography

The recombinant catalytic polypeptides or proteolytic antibody light chains of the present invention can also be separated from other proteins on the basis of their size, net surface charge, hydrophobicity, and affinity for ligands. In addition, antibodies raised against recombinant polypeptides or the proteolytic light chains (e.g., those comprising the amino acid sequence of SEQ ID NO:2, 4, 6, 8, 10, 12, 14, 16, 18, 20, 22, 24, 26, or 28) can be conjugated to column matrices and the polypeptides can thus be immunopurified. All of these methods are well known in the art.

It will be apparent to one of skill that chromatographic techniques can be performed at any scale and using equipment from many different manufacturers (e.g., Pharmacia Biotech).

V. Operably Joining Antibody Light Chain and Antibody Heavy Chain

There are several methods to join the antibody light chain and heavy chain of the recombinant catalytic polypeptides. For example, one skilled in the art will recognize that when genes encoding two antibody chains are expressed in transfected cells simultaneously, they will be joined during the process. The two antibody chains may also be joined at nucleic acid level or at polypeptide level, before or after their expression.

A. Recombinant Methods

An antibody light chain and an antibody heavy chain can be joined by recombinant DNA technology prior to their expression (see, e.g., Chaudhary et al, Nature, 339:394-397 (1989); Pantoliano et al., Biochemistry, 30:10117-10125 (1991); Kim et al., Mol. Immunol., 34:891-906 (1997)). As a person of ordinary skill in the art will know, a polynucleotide sequence can be introduced to connect the coding sequences for the antibody light and heavy chains by employing various tools and techniques such as enzymatic digestion/ligation and/or PCR. The precise length of the insertion is essential in that the open reading frame of the coding sequence down stream from the insertion should not be disrupted. Upon transfection and expression, one single polypeptide is generated, which contains both the antibody light and heavy chains and a peptide linker of appropriate length joining them.

A second approach in joining the antibody light and heavy chains also takes advantage of the recombinant DNA technology, although the two antibody chains remain two separate polypeptides when expressed. In this approach, nucleotide sequences encoding suitable tags are fused to the 3′ ends of the genes encoding the antibody chains. Upon transfection and expression of the tagged antibody chains, they can be attached to a common solid support, to which appropriate tag-binders have already been immobilized. The antibody chains are thus joined via the solid support by virtue of being within close physical proximity. The general methodology of making fusion proteins is well known to those skilled in the art and instructions can be found in many scientific publications such as Sambrook and Russell, supra. A number of tags and tag-binders that can be attached to solid support are known to skilled artisans based on molecular interactions well described in the literature. Suitable pairs for this purpose include biotin and avidin or streptavidin, the Fc region of an antibody and protein A or protein G, etc. Further, a large number of known cell surface receptor-ligand pairs can also be useful, e.g., cytokines, cell adhesion molecules, viral proteins, steroids, and various toxins/venoms with their respective receptors. Many of these tags or their coding sequences are commercially available.

Derived from the second approach, a third approach involves fusing a nucleotide sequence encoding a tag (or a ligand) to a first antibody chain and a nucleotide sequence encoding a tag-binder (or receptor) to a second antibody chain. The two antibody chains can thus be joined via the interaction of the tag and the tag-binder (or the ligand and the receptor) without the aid of solid support.

B. Chemical Methods

The two antibody chains may also be joined by chemical means following their expression and purification. Chemical modifications include, for example, derivitization for the purpose of linking the antibody chains to each other, either directly or through a linking compound, by methods that are well known in the art of protein chemistry. Both covalent and noncovalent attachment means may be used with the recombinant catalytic polypeptides of the present invention.

The procedure for linking the two antibody chains will vary according to the chemical structure of the moieties where the chains are joined. As a polypeptide one antibody chain typically contain a variety of functional groups such as carboxylic acid (—COOH), free amine (—NH₂), or sulfhydryl (—SH) groups, which are available for reaction with a suitable functional group on the other antibody chain to result in a linkage.

Alternatively, one antibody chain can be derivatized to expose or to attach additional reactive functional groups. The derivatization may involve attachment of any of a number of linker molecules such as those available from Pierce Chemical Company, Rockford Ill. The linker is capable of forming covalent bonds to both antibody chains. Suitable linkers are well known to those of skill in the art and include, but are not limited to, straight or branched-chain carbon linkers, heterocyclic carbon linkers, or peptide linkers. Since the antibody chains are polypeptides, the linkers may be joined to the constituent amino acids through their side groups (for example, through a disulfide linkage to cysteine). The linkers may also be joined to the alpha carbon amino and carboxyl groups of the terminal amino acids.

As discussed in the last section, the antibody chains can be joined via the interaction of a tag and a tag-binder. The tags and tag-binders can be attached to the antibody chains by chemical means. For example, synthetic polymers, such as polyurethanes, polyesters, polycarbonates, polyureas, polyamides, polyethyleneimines, polyarylene sulfides, polysiloxanes, polyimides, and polyacetates can form an appropriate tag or tag binder. Other common linkers such as peptides, polyethers, and the like can also serve as tags, and include polypeptide sequences, such as poly-Gly sequences of between about 5 and 200 amino acids (SEQ ID NO:62). Such flexible linkers are known to persons of skill in the art. For example, poly(ethylene glycol) linkers are available from Shearwater Polymers, Inc. Huntsville, Ala. These linkers optionally have amide linkages, sulfhydryl linkages, or heterofunctional linkages. Many additional tag/tag binder pairs can also be used for this purpose and would be apparent to one of skill upon review of this disclosure.

Alternatively, the antibody chains can be joined via tag/tag-binder interaction when one of the binding parties is first immobilized to a solid support. Tag binders are fixed to solid substrates using any of a variety of methods currently available. Solid substrates are commonly derivatized or functionalized by exposing all or a portion of the substrate to a chemical reagent which fixes a chemical group to the surface which is reactive with a portion of the tag binder. For example, groups which are suitable for attachment to a longer chain portion would include amines, hydroxyl, thiol, and carboxyl groups. Aminoalkylsilanes and hydroxyalkylsilanes can be used to functionalize a variety of surfaces, such as glass surfaces. The construction of such solid phase biopolymer arrays is well described in the literature. See, e.g., Merrifield, J. Am. Chem. Soc. 85:2149-2154 (1963) (describing solid phase synthesis of, e.g., peptides); Geysen et al., J. Immun. Meth. 102:259-274 (1987) (describing synthesis of solid phase components on pins); Frank & Doring, Tetrahedron 44:6031-6040 (1988) (describing synthesis of various peptide sequences on cellulose disks); Fodor et al., Science, 251:767-777 (1991); Sheldon et al., Clinical Chemistry 39(4):718-719 (1993); and Kozal et al., Nature Medicine 2(7):753759 (1996) (all describing arrays of biopolymers fixed to solid substrates). Non-chemical approaches for fixing tag binders to substrates include other common methods, such as heat, cross-linking by UV radiation, and the like.

C. Cellular Methods

Hybridoma cells can be generated by fusing B cells producing a desired antibody with an immortalized cell line, usually a myeloma cell line, so that the resulting fusion cells will be an immortalized cell line that secretes a particular antibody. By the same principle, myeloma cells can be first transfected with a nucleic acid encoding a proteolytic light chain (e.g., a nucleic acid comprising a nucleotide sequence of SEQ ID NO: 1, 3, 5, 7, 9, 11, 13, 15, 17, 19, 21, 23, 25, or 27) and can be screened for the expression of the light chain. Those myeloma cells with highest level of proteolytic light chain expression can be subsequently fused with B cells that produce an antibody with desired target protein specificity. The fusion cells will produce two types of antibodies: one is a heterologous antibody containing a heterologous heavy chain operably joined to the catalytic light chain (e.g., one comprising an amino acid sequence of SEQ ID NO:2, 4, 6, 8, 10, 12, 14, 16, 18, 20, 22, 24, 26, or 28), and the other is the same antibody that the parental B cells would secrete. The operably joined heterologous heavy and light chains can be isolated by conventional methods such as chromatography and the enzymatic activity can be confirmed by target protein binding assays and endopeptidase activity assays described in other sections of this disclosure. Alternatively, the gene for the heavy chain may be cloned from the fused cell by standard techniques and used to express in other mammalian cell lines operably linked with the catalytic light chain. In some cases, where the heterologous antibody is the predominant type in quantity among the two types of antibodies, such isolation may not be needed.

VI. In Vitro Enzymatic Activity of Recombinant Catalytic Polypeptides

A. Target Protein Binding Assays

The ability to specifically bind a target protein by a recombinant catalytic polypeptide of the present invention or an antibody heavy chain thereof can be demonstrated in a variety of in vitro assays utilizing techniques one of ordinary skill in the art would be familiar with. The general principles and methodologies of these assays are the same as that of immunoassays designed for detecting target protein levels in patient samples, which are described in detail in a later section.

For example, to screen for target protein specificities in a library of recombinant catalytic polypeptides, which are in the form of single polypeptide chains containing both antibody light and heavy chains, target proteins can be bound directly to a solid substrate and thus immobilized in an assay system. Recombinant polypeptides obtained from an expression system such as in a phage display library can be labeled, for example, with ¹²⁵I, and can be easily detected when captured by the target proteins. Signal comparison with an irrelevant antibody (i.e., one that is known not to bind the target protein) labeled, for example, with ¹²⁵I, will reveal whether a particular recombinant polypeptide is specific for the target protein. Phage display can be utilized directly for this purpose: phages containing a recombinant polypeptide with specificity for a target protein are first bound to the target protein already immobilized to a solid phase. They are subsequently washed off under stringent conditions (such as washing with detergent, a high salt solution, or low pH) and recovered. The recovered fraction can be tested for multiplicity of infection (MOI) in comparison with negative control phages processed in the same manner to determine specificity. Alternatively, recombinant polypeptides may be immobilized and the target proteins labeled for a screening assay. Specific antibodies should show statistically significant signals above background in the above assays, and such signals are preferably at least two fold above the background. A variety of other methods are also available and will be obvious for a skilled artisan to employ to identify a recombinant catalytic polypeptide with specificity for a target protein.

The same general methods are applicable for screening and selecting individual antibody heavy chains for target protein specificity prior to being joined with a proteolytic light chain, as well as for confirming the binding specificity of a recombinant catalytic polypeptide after a proteolytic light chain and a selected heavy chain are operably joined.

B. Endopeptidase Activity Assay

(1) Hydrolysis of Peptide by Antibody Light Chain

Several assays are available to determine whether an antibody light chain contains endopeptidase activity. Generally, any assay that can detect hydrolysis of a secondary amide bond may be used to determine endopeptidase activity. Commonly used assays utilize peptide analogs conjugated to reporter molecules that can be detected when released from the peptide. A commonly used assay involves a peptide-methylcoumarinamide (MCA) derivative, such that hydrolysis of the peptide-MCA bond produces the leaving group aminomethylcoumarin whose fluorescence is measured at an excitation of 370 nm and an emmission of 460 nm. Such an assay has been practiced to detect proteolytic activity of murine light chains (Gao, et al, J Biol. Chem. 269:32389-32393 (1994); Sun et al, J. Mol. Biol. 271:374-385 (1997)). Other similar methods are known in the art to conjugate peptides to molecules that have altered spectral properties when they are cleaved (e.g., nitroaniline conjugates).

(2) Hydrolysis of Target Protein by Recombinant Catalytic Polypeptide

Any method that allows detection of a cleaved peptide bond in a target protein is suitable for use in the present invention. Since hydrolysis of a peptide bond necessarily produces more that one polypeptide product, several standard size or mass analysis techniques well known in the art can be used to identify peptide bond hydrolysis. These techniques include electrophoretic mobility techniques such as SDS polyacrylamide gel electrophoresis, high performance liquid chromatography (HPLC), and mass spectrometry methods such as MALDI-TOF. Alternatively, a protein labeled with a radioisotope can be precipitated in TCA, wherein hydrolysis of a peptide bond will be indicated by the amount of TCA soluble radioactivity (Gao, et al, J. Biol. Chem. 269: 32389-32393 (1994)). Other methods for detecting target protein hydrolysis include coupling a labeled target protein to a solid support, and measuring release of the labeled protein following exposure to the catalytic polypeptide. Furthermore, Smith and Kohom (PNAS 88: 5159-5162 (1991)), Lawler and Snyder (Anal. Biochem. 269: 133-138 (1999)), Dasmahaptra, et al (PNAS 89: 4159-4162(1992)), Murray, et al (Gene 134: 123-128 (1993)), and Kim, et al (Biochem.Biophys.Res. Commun. 296: 419 (2002)) describe genetic mechanisms for detecting proteolytic activity using variants of the yeast two-hybrid system. This system could be modified to accommodate recombinant catalytic polypeptides of the present invention.

(3) Binding of Recombinant Catalytic Polypeptides to Protease Inhibitor Probes

A functional recombinant catalytic polypeptide can be assayed be its ability to bind to a protease inhibitor probe. A “protease inhibitor probe” in the context of the present invention refers to a bifunctional molecule comprising a protease inhibitor component and a detectible ligand component. A protease inhibitor component may be any inhibitor that can functionally inhibit a serine protease. Such inhibitors include small molecules and derivatives thereof including phosphonates like diisopropyl fluorophosphate (DFP), or phenylmethylsulfonylfluoride (PMSF) as well as protein or peptide inhibitors such as aprotinin and the like. Preferably the inhibitor can covalently bind to one of the components of a serine protease triad. Recent work has shown that fluorophosphonate probes could be used to profile proteins with hydrolase activity in complex proteomic mixtures (Liu, et al. Proc. Natl. Acad. Sci. 96: 14694-14699 (1999)). Recombinant catalytic polypeptides could also be identified using covalently reactive analogs which are phosphonate esters (Paul, et al. J. Biol. Chem. 276: 28314-28320 (2001)).

Examples of detectible ligands (including labels) useful in a protease inhibitor probe, include, but are not limited to, biotin, deiminobiotin, dethiobiotin, vicinal diols, such as 1,2-dihydroxyethane, 1,2-dihydroxycyclohexane, etc., digoxigenin, maltose, oligohistidine, glutathione, 2,4-dintrobenzene, phenylarsenate, ssDNA, dsDNA, a peptide of polypeptide, a metal chelate, a saccharide, rhodamine or fluorescein, or any hapten to which an antibody can be generated. A detectable label is a group that is detectable at low concentrations, usually less than micromolar, preferably less than nanomolar, that can be readily distinguished from other analogous molecules, due to differences in molecular weight, redox potential, electromagnetic properties, binding properties, and the like. The detectable label may be a hapten, such as biotin, or a fluorescer, or an oligonucleotide, capable of non-covalent binding to a complementary receptor other than the active protein; a mass tag comprising a stable isotope; a radioisotope; a metal chelate or other group having a heteroatom not usually found in biological samples; a fluorescent or chemiluminescent group preferably having a quantum yield greater than 0.1; an electroactive group having a lower oxidation or reduction potential than groups commonly present in proteins; a catalyst such as a coenzyme, organometallic catalyst, photosensitizer, or electron transfer agent; a group that affects catalytic activity such as an enzyme activator or inhibitor or a coenzyme.

VII. In Vivo Target Protein Cleavage by Recombinant Catalytic Polypeptides

A. Administration of Recombinant Catalytic Polypeptides

The recombinant catalytic polypeptides of the present invention can be administered directly to a mammalian subject for specific hydrolysis of target proteins in vivo. Diseases and conditions that can be treated or prevented using this strategy include those involving overexpression of a normal protein or expression of an aberrant protein, or where a foreign protein plays a role in the pathogenesis of the disease or condition; they can be inherited or acquired in nature. Cancers of various types, allergic reactions, viral and bacterial infections are some examples. In some embodiments, recombinant catalytic polypeptides of the present invention can be combined with other drugs useful for relieving certain symptoms of the diseases.

(1) Pharmaceutical Formulations

The pharmaceutical compositions containing recombinant catalytic polypeptides of the present invention may comprise a pharmaceutically acceptable carrier. Pharmaceutically acceptable carriers are determined in part by the particular composition being administered, as well as by the particular method used to administer the composition. Accordingly, there is a wide variety of suitable formulations of pharmaceutical compositions of the present invention (see, e.g., Remington's Pharmaceutical Sciences, Mack Publishing Company, Philadelphia, Pa., 19th ed. 1995).

The recombinant catalytic polypeptides of the present invention, alone or in combination with other suitable components, can be made into aerosol formulations (i.e., they can be “nebulized”) to be administered via inhalation or in compositions useful for injection. Aerosol formulations can be placed into pressurized acceptable propellants, such as dichlorodifluoromethane, propane, nitrogen, and the like.

Formulations suitable for administration include aqueous and non-aqueous solutions, isotonic sterile solutions, which can contain antioxidants, buffers, bacteriostats, and solutes that render the formulation isotonic, and aqueous and non-aqueous sterile suspensions that can include suspending agents, solubilizers, thickening agents, stabilizers, and preservatives. In the practice of this invention, compositions can be administered, for example, orally, nasally, topically, intravenously, intraperitoneally, or intrathecally. The formulations of compounds can be presented in unit-dose or multi-dose sealed containers, such as ampoules and vials. Solutions and suspensions can be prepared from sterile powders, granules, and tablets of the kind previously described. The modulators can also be administered as part of a prepared food or drug.

(2) Administration and Dosage

Administration of compositions containing recombinant catalytic polypeptides of the invention can be by any of the routes normally used for introducing a therapeutic compound into ultimate contact with the tissue to be treated and is well known to those of skill in the art. As mentioned above, various methods are available for administering a composition to a mammal. Modes of administration may include, but are not limited to, methods that involve administering the composition intravenously, intraperitoneally, intranasally, transdermally, topically, subcutaneously, parentally, intramuscularly, orally, or systemically, and via injection, ingestion, inhalation, implantation, or adsorption by any other means. Although more than one route can be used to administer a particular composition, a particular route can often provide a more immediate and more effective reaction than another route.

The dose of a recombinant catalytic polypeptide administered to a mammalian patient, in the context of the present invention, should be sufficient to effect a beneficial response, i.e., to reduce the level of a target protein, in the patient over time. The optimal dose level for any patient will depend on a variety of factors including the efficacy of the specific recombinant catalytic polypeptide employed, the age, body weight, physical activity, and diet of the patient, on a possible combination with other drugs, and on the severity of the disease to be treated. The size of the dose will also be determined by the existence, nature, and extent of any adverse side-effects that accompany the administration of a particular compound or vector in a particular subject.

In determining the effective amount of the recombinant catalytic polypeptide to be administered a physician may evaluate circulating plasma levels of the recombinant polypeptide, polypeptide toxicity, and the production of anti-polypeptide antibodies. In general, the dose equivalent of a recombinant catalytic polypeptide is from about 1 pg-10 mg/kg for a typical subject. The administration of the recombinant catalytic peptides can be one time or multiple times over the course of treatment.

For administration, recombinant catalytic polypeptides of the present invention can be administered at a rate determined by the LD-50 of the polypeptides, and the side-effects of the polypeptides at various concentrations, as applied to the mass and overall health of the subject. Administration can be accomplished via single or divided doses.

B. Administration of Nucleic Acids Encoding Recombinant Catalytic Polypeptides

Similar to administration of recombinant catalytic polypeptides for treatment or prevention of a variety of human diseases and conditions, nucleic acids encoding such recombinant polypeptides can be administered directly to a mammalian subject.

(1) Vectors for Gene Delivery

For delivery to a cell or organism, the nucleic acids encoding recombinant catalytic polypeptides can be incorporated into a vector. Examples of vectors used for such purposes include expression plasmids capable of directing the expression of the nucleic acids in the target cell. In other instances, the vector is a viral vector system wherein the nucleic acids are incorporated into a viral genome that is capable of transfecting the target cell. In a preferred embodiment, the nucleic acids can be operably linked to expression and control sequences that can direct expression of the gene in the desired target host cells. Thus, one can achieve expression of the nucleic acid under appropriate conditions in the target cells.

(2) Gene Delivery Systems

Viral vector systems useful in the expression of the nucleic acids encoding recombinant catalytic polypeptides include, for example, naturally-occurring or recombinant viral vector systems. Depending upon the particular application, suitable viral vectors include replication competent, replication deficient, and conditionally replicating viral vectors. For example, viral vectors can be derived from the genome of human or bovine adenoviruses, vaccinia virus, herpes virus, adeno-associated virus, minute virus of mice (MVM), HIV, sindbis virus, and retroviruses (including but not limited to Rous sarcoma virus), and MoMLV. Typically, the genes of desired recombinant catalytic polypeptides are inserted into such vectors to allow packaging of the gene construct, typically with accompanying viral DNA, followed by infection of a sensitive host cell and expression of the polypeptide.

As used herein, “gene delivery system” refers to any means for the delivery of a nucleic acid encoding a recombinant catalytic polypeptide to a target cell. In some embodiments of the invention, the nucleic acids are conjugated to a cell receptor ligand for facilitated uptake (e.g., invagination of coated pits and internalization of the endosome) through an appropriate linking moiety, such as a DNA linking moiety (Wu et al., J. Biol. Chem., 263:14621-14624 (1988)); WO 92/06180). For example, nucleic acids can be linked through a polylysine moiety to asialo-oromucocid, which is a ligand for the asialoglycoprotein receptor of hepatocytes.

Similarly, viral envelopes used for packaging gene constructs that include the nucleic acids encoding recombinant catalytic polypeptides can be modified by the addition of receptor ligands or antibodies specific for a receptor to permit receptor-mediated endocytosis into specific cells (see, e.g., WO 93/20221, WO 93/14188, and WO 94/06923). In some embodiments of the invention, DNA constructs containing nucleic acids encoding recombinant catalytic polypeptides are linked to viral proteins, such as adenovirus particles, to facilitate endocytosis (Curiel et al., Proc. Natl. Acad. Sci. U.S.A., 88:8850-8854 (1991)). In other embodiments, molecular conjugates containing nucleic acids encoding recombinant catalytic polypeptides can include microtubule inhibitors (WO 94/06922), synthetic peptides mimicking influenza virus hemagglutinin (Plank et al., J. Biol. Chem., 269:12918-12924 (1994)), and nuclear localization signals such as SV40 T antigen (WO 93/19768).

Retroviral vectors are also useful for introducing the nucleic acids encoding recombinant catalytic polypeptides into target cells or organisms. Retroviral vectors are produced by genetically manipulating retroviruses. The viral genome of retroviruses is RNA. Upon infection, this genomic RNA is reverse transcribed into a DNA copy which is integrated into the chromosomal DNA of transduced cells with a high degree of stability and efficiency. The integrated DNA copy is referred to as a provirus and is inherited by daughter cells as is any other gene. The wild type retroviral genome and the proviral DNA have three genes: the gag, the pol, and the env genes, which are flanked by two long terminal repeat (LTR) sequences. The gag gene encodes the internal structural (nucleocapsid) proteins; the pol gene encodes the RNA directed DNA polymerase (reverse transcriptase); and the env gene encodes viral envelope glycoproteins. The 5′ and 3′ LTRs serve to promote transcription and polyadenylation of virion RNAs. Adjacent to the 5′ LTR are sequences necessary for reverse transcription of the genome (the tRNA primer binding site) and for efficient encapsulation of viral RNA into particles (the Psi site) (see, Mulligan, Experimental Manipulation of Gene Expression, Inouye (ed), pp 155-173 (1983)); Mann et al., Cell, 33:153-159 (1983)); Cone and Mulligan, Proc. Natl. Acad. Sci. USA, 81:6349-6353 (1984)).

The design of retroviral vectors is well known to those of ordinary skill in the art. In brief, if the sequences necessary for encapsidation (or packaging of retroviral RNA into infectious virions) are missing from the viral genome, the result is a cis-acting defect which prevents encapsidation of genomic RNA. However, the resulting mutant is still capable of directing the synthesis of all virion proteins. Retroviral genomes from which these sequences have been deleted, as well as cell lines containing the mutant genome stably integrated into the chromosome are well known in the art and are used to construct retroviral vectors. Preparation of retroviral vectors and their uses are described in many publications including, e.g., European Patent Application EPA 0 178 220; U.S. Pat. No. 4,405,712; Gilboa, Biotechniques, 4:504-512 (1986); Mann et al., supra; Cone and Mulligan, supra; Eglitis et al, Biotechniques, 6:608-614 (1988); Miller et al., Biotechniques, 7:981-990 (1989); Miller (1992) supra; Mulligan (1993), supra; and WO 92/07943.

The retroviral vector particles are prepared by recombinantly inserting the desired nucleotide sequence into a retrovirus vector and packaging the vector with retroviral capsid proteins by use of a packaging cell line. The resultant retroviral vector particle is incapable of replication in the host cell but is capable of integrating into the host cell genome as a proviral sequence containing the desired nucleotide sequence. As a result, the patient is capable of producing, for example, a DNA sequence and subsequently a recombinant catalytic polypeptide of the present invention and thus catalyze the cleavage of the target protein.

Packaging cell lines that are used to prepare the retroviral vector particles are typically recombinant mammalian tissue culture cell lines that produce the necessary viral structural proteins required for packaging, but which are incapable of producing infectious virions. The defective retroviral vectors that are used, on the other hand, lack these structural genes but encode the remaining proteins necessary for packaging. To prepare a packaging cell line, one can construct an infectious clone of a desired retrovirus in which the packaging site has been deleted. Cells comprising this construct will express all structural viral proteins, but the introduced DNA will be incapable of being packaged. Alternatively, packaging cell lines can be produced by transforming a cell line with one or more expression plasmids encoding the appropriate core and envelope proteins. In these cells, the gag, pol, and env genes can be derived from the same or different retroviruses.

A number of packaging cell lines suitable for the present invention are also available in the prior art. Examples of these cell lines include Crip, GPE86, PA317, and PG13 (see Miller et al., J. Virol., 65:2220-2224 (1991)). Examples of other packaging cell lines are described in Cone and Mulligan, supra; Danos and Mulligan, Proc. Natl. Acad. Sci. USA, 85:6460-6464 (1988); Eglitis et al. (1988), supra; and Miller (1990), supra.

Packaging cell lines capable of producing retroviral vector particles with chimeric envelope proteins may be used. Alternatively, amphotropic or xenotropic envelope proteins, such as those produced by PA317 and GPX packaging cell lines may be used to package the retroviral vectors.

(3) Pharmaceutical Formulations

When used for pharmaceutical purposes, the vectors used for therapy involving nucleic acid transfer are formulated in a suitable buffer, which can be any pharmaceutically acceptable buffer, such as phosphate buffered saline or sodium phosphate/sodium sulfate, Tris buffer, glycine buffer, sterile water, and other buffers known to the ordinarily skilled artisan such as those described by Good et al., Biochemistry, 5:467 (1966).

The compositions can additionally include a stabilizer, enhancer, or other pharmaceutically acceptable carriers or vehicles. A pharmaceutically acceptable carrier can contain a physiologically acceptable compound that acts, for example, to stabilize the nucleic acids encoding recombinant catalytic polypeptides and any associated vector. A physiologically acceptable compound can include, for example, carbohydrates, such as glucose, sucrose, or dextrans; antioxidants, such as ascorbic acid or glutathione; chelating agents; low molecular weight proteins or other stabilizers or excipients. Other physiologically acceptable compounds include wetting agents, emulsifying agents, dispersing agents, or preservatives, which are particularly useful for preventing the growth or action of microorganisms. Various preservatives are well known and include, for example, phenol and ascorbic acid. Examples of carriers, stabilizers, or adjuvants can be found in Remington's Pharmaceutical Sciences, supra.

(4) Administration and Dosage

The formulations containing the nucleic acids of the invention can be delivered to any tissue or organ using any delivery method known to the ordinarily skilled artisan. In some embodiments of the invention, the nucleic acids of the invention are formulated in mucosal, topical, and/or buccal formulations, particularly mucoadhesive gel and topical gel formulations. Exemplary permeation-enhancing compositions, polymer matrices, and mucoadhesive gel preparations for transdermal delivery are disclosed in U.S. Pat. No. 5,346,701.

Effective dosage of the formulations will vary depending on many different factors, including means of administration, target sire, physiological state of the patient, and other medicines administered. Thus, treatment dosages will need to be titrated to optimize safety and efficacy. In determining the effective amount of the vector to be administered, the physician should evaluate the particular nucleic acid used, the disease state being diagnosed; the age, weight, and overall condition of the patient, circulating plasma levels, vector toxicities, progression of the disease, and the production of anti-vector antibodies. The size of the dose also will be determined by the existence, nature, and extent of any adverse side-effects that accompany the administration of a particular vector. To practice the present invention, doses ranging from about 10 ng-1 g, 100 ng-100 mg, 1 μg-10 mg, or 30-300 μg DNA per patient are typical. Doses generally range between about 0.01 and about 50 mg per kilogram of body weight, preferably between about 0.1 and about 5 mg/kg of body weight or about 10⁸10¹⁰ or 10¹² particles per injection. In general, the dose equivalent of a naked nucleic acid from a vector is from about 1 μg-100 μg for a typical 70 kg patient, and doses of vectors which include a retroviral particle are calculated to yield an equivalent amount of nucleic acid encoding a recombinant catalytic polypeptide.

(5) Methods of Treatment

The gene therapy formulations of the invention are typically administered to a cell. The cell can be provided as part of a tissue, such as an epithelial membrane, or as an isolated cell, such as in tissue culture. The cell can be provided in vivo, ex vivo, or in vitro.

The formulations can be introduced into the tissue of concern in vivo or ex vivo by a variety of methods. In some embodiments, the nucleic acids encoding recombinant catalytic polypeptides are introduced into cells by such methods as microinjection, calcium phosphate precipitation, liposome fusion, or biolistics. In further embodiments, the nucleic acids are taken up directly by the tissue of concern.

In some embodiments, the nucleic acids encoding recombinant catalytic polypeptides are administered ex vivo to cells or tissues explanted from a patient, then returned to the patient. Examples of ex vivo administration of therapeutic gene constructs include Nolta et al., Proc Natl. Acad. Sci. USA, 93:2414-2419 (1996); Koc et al., Seminars in Oncology, 23:46-65 (1996); Raper et al., Annals of Surgery, 223:116-126 (1996); Dalesandro et al., J. Thorac. Cardi. Surg., 11:416-422 (1996); and Makarov et al., Proc. Natl. Acad. Sci. USA, 93:402-406 (1996).

C. Detection of Target Protein Reduction In Vivo

Following the administration of therapeutic compounds containing either recombinant catalytic polypeptides or nucleic acids encoding recombinant catalytic polypeptides, the effectiveness of the therapeutic compounds can be assessed by comparing the in vivo target protein level before and after the administration.

The general methods of measuring protein levels in tissue samples are well known to ordinarily skilled artisans. As mentioned above, various immunoassays are routinely used to detect a protein of interest. A general overview of the applicable technology can be found in Harlow and Lane, Antibodies, A Laboratory Manual, 1988.

(1) Antibodies to Target Proteins

Methods for producing polyclonal and monoclonal antibodies that react specifically with a target protein are known to those of skill in the art (see, e.g., Coligan, supra; and Harlow and Lane, supra; Stites et al., supra and references cited therein; Goding, supra; and Kohler and Milstein, Nature, 256:495-497 (1975)). For example, to produce polyclonal antibodies, a purified target protein is mixed with an adjuvant and used to immunize animals. When high titers of antibody to the target protein are obtained, blood is collected from the animals and antisera are prepared for immunoassays. To produce monoclonal antibodies, spleen cells from an animal immunized with a target protein are immortalized, commonly by fusion with a myeloma cell (see, Kohler and Milstein, Eur. J Immunol., 6:511-519 (1976)). Colonies arising from single immortalized cells are screened for production of antibodies of the desired specificity and affinity for the target protein.

(2) Immunoassays

Once antibodies specific for a target protein are available, the target protein level in a patient can be measured by a variety of immunoassay methods with qualitative and quantitative results available to the clinician. Various samples from the patient, such as blood, urine, or tissue, can be used in the immunoassays to detected the in vivo target protein level, depending on the particular disease to be treated. For a review of immunological and immunoassay procedures in general see, e.g., Stites, supra; U.S. Pat. Nos. 4,366,241; 4,376,110; 4,517,288; and 4,837,168.

i. Labeling in Immunoassays

Immunoassays often utilize a labeling agent to specifically bind to and label the binding complex formed by the antibody and the target protein. The labeling agent may itself be one of the moieties comprising the antibody/target protein complex, or may be a third moiety, such as another antibody, that specifically binds to the antibody/target protein complex. A label may be detectable by spectroscopic, photochemical, biochemical, immunochemical, electrical, optical or chemical means. Some examples are, but not limited to, magnetic beads (e.g., Dynabeads™), fluorescent dyes (e.g., fluorescein isothiocyanate, Texas red, rhodamine, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold or colored glass or plastic (e.g., polystyrene, polypropylene, latex, etc.) beads.

In some cases, the labeling agent is a second antibody bearing a label. Alternatively, the second antibody may lack a label, but it may, in turn, be bound by a labeled third antibody specific to antibodies of the species from which the second antibody is derived. The second antibody can be modified with a detectable moiety, such as biotin, to which a third labeled molecule can specifically bind, such as enzyme-labeled streptavidin.

Other proteins capable of specifically binding immunoglobulin constant regions, such as protein A or protein G, can also be used as the label agents. These proteins are normal constituents of the cell walls of streptococcal bacteria. They exhibit a strong non-immunogenic reactivity with immunoglobulin constant regions from a variety of species (see, generally, Kronval, et al. J. Immunol., 111:1401-1406 (1973); and Akerstrom, et al., J. Immunol., 135:2589-2542 (1985)).

ii. Immunoassay Formats

Immunoassays for detecting target proteins from tissue samples may be either competitive or noncompetitive. Noncompetitive immunoassays are assays in which the amount of captured target protein is directly measured. In one preferred “sandwich” assay, for example, the antibody specific for the target protein can be bound directly to a solid substrate where the antibody is immobilized. It then captures the target protein in test samples. The antibody/target protein complex thus immobilized is then bound by a labeling agent, such as a second antibody bearing a label. Alternatively, the second antibody may lack a label, but it may, in turn, be bound by a labeled third antibody specific to antibodies of the species from which the second antibody is derived. The second can be modified with a detectable moiety, such as biotin, to which a third labeled molecule can specifically bind, such as enzyme-labeled streptavidin.

In competitive assays, the amount of target protein in a sample is measured indirectly by measuring the amount of an added (exogenous) target protein displaced (or competed away) from an antibody specific for the target protein by the target protein present in the sample. In a typical example of such an assay, the antibody is immobilized and the exogenous target protein is labeled. Since the amount of the exogenous target protein bound to the antibody is inversely proportional to the concentration of the target protein present in the sample, the target protein level in the sample can thus be determined based on the amount of exogenous target protein bound to the antibody and thus immobilized.

In some cases, western blot (immunoblot) analysis is used to detect and quantify the presence of a target protein in the samples from a patient. The technique generally comprises separating sample proteins by gel electrophoresis on the basis of molecular weight, transferring the separated proteins to a suitable solid support (such as a nitrocellulose filter, a nylon filter, or a derivatized nylon filter) and incubating the samples with the antibodies that specifically bind the target protein. These antibodies may be directly labeled or alternatively may be subsequently detected using labeled antibodies (e.g., labeled sheep anti-mouse antibodies) that specifically bind to the antibodies against the target protein.

Other assay formats include liposome immunoassays (LIA), which use liposomes designed to bind specific molecules (e.g., antibodies) and release encapsulated reagents or markers. The released chemicals are then detected according to standard techniques (see, Monroe et al., Amer. Clin. Prod. Rev., 5:34-41 (1986)).

VIII. Libraries of Recombinant Catalytic Polypeptides

A. Display Libraries

Libraries of recombinant catalytic polypeptides of the present invention can be constructed using a number of different display systems. In cell or virus-based systems, the recombinant polypeptides can be displayed, for example, on the surface of a particle, e.g., a virus or cell and screened for the ability to specifically bind and cleave a target protein. In vitro display systems can also be used, in which the recombinant polypeptides are linked to an agent that provides a mechanism for coupling a recombinant polypeptide to the nucleic acid sequence that encodes it. These technologies include ribosome display and mRNA display.

In some instances, for example, ribosomal display, a recombinant catalytic polypeptide is linked to the encoding nucleic acid sequence through a physical interaction, for example, with a ribosome. In other embodiments, e.g., mRNA display, a recombinant catalytic polypeptide may be joined to another molecule via a linking group. The linking group can be a chemical crosslinking agent, including, for example, succinimidyl-(N-maleimidomethyl)-cyclohexane-1-carboxylate (SMCC). The linking group can also be an additional amino acid sequence(s), including, for example, a polyalanine, polyglycine or similar linking group. Other near neutral amino acids, such as Ser can also be used in the linker sequence. Amino acid sequences which may be usefully employed as linkers include those disclosed in Maratea et al. Gene 40:39-46 (1985); Murphy et al. Proc. Natl. Acad. Sci. USA 83:8258-8262 (1986); U.S. Pat. Nos. 4,935,233 and 4,751,180. The linker sequence may generally be from 1 to about 50 amino acids in length, e.g., 2, 3, 4, 6, or 10 amino acids in length, but can be 100 or 200 amino acids in length.

Other chemical linkers include carbohydrate linkers, lipid linkers, fatty acid linkers, polyether linkers, e.g., PEG, etc. For example, poly(ethylene glycol) linkers are available from Shearwater Polymers, Inc. Huntsville, Ala. These linkers optionally have amide linkages, sulfhydryl linkages, or heterofunctional linkages.

(1) Phage Display Libraries

Construction of phage display libraries exploits the bacteriophage's ability to display peptides and proteins on their surfaces, i.e., on their capsids. Often, filamentous phage such as M13, fd, or fl are used. Filamentous phage contain single-stranded DNA surrounded by multiple copies of genes encoding major and minor coat proteins, e.g., pIII. Coat proteins are displayed on the capsid's outer surface. DNA sequences inserted in-frame with capsid protein genes are co-transcribed to generate fusion proteins or protein fragments displayed on the phage surface. Phage libraries thus can display polypeptides representative of the diversity of the inserted sequences. Significantly, these polypeptides can be displayed in “natural” folded conformations. The recombinant catalytic polypeptides expressed on phage display libraries can then specifically bind and cleave target proteins.

The concept of using filamentous phages, such as M13 or fd, for displaying polypeptides on phage capsid surfaces was first introduced by Smith, Science 228:1315-1317 (1985). Polypeptides have been displayed on phage surfaces to identify many potential ligands (see, e.g., Cwirla, Proc. Natl. Acad. Sci. USA 87:6378-6382 (1990)). There are numerous systems and methods for generating phage display libraries described in the scientific and patent literature, see, e.g., Sambrook and Russell, Molecule Cloning: A Laboratory Manual, 3rd edition, Cold Spring Harbor Laboratory Press, Chapter 18, (2001); Phage Display of Peptides and Proteins: A Laboratory Manual, Academic Press, San Diego, 1996; Crameri, Eur. J. Biochem. 226:53-58 (1994); de Kruif, Proc. Natl. Acad. Sci. USA 92:3938-3942 (1995); McGregor, Mol. Biotechnol. 6:155-162 (1996); Jacobsson, Biotechniques 20:1070-1076 (1996); Jespers, Gene 173:179-181 (1996); Jacobsson, Microbiol Res. 152:121-128 (1997); Fack, J. Immunol. Methods 206:43-52 (1997); Rossenu, J. Protein Chem. 16:499-503 (1997); Katz, Annu. Rev. Biophys. Biomol. Struct. 26:27-45 (1997); Rader, Curr. Opin. Biotechnol. 8:503-508 (1997); Griffiths, Curr. Opin. Biotechnol. 9:102-108 (1998).

Typically, exogenous nucleic acids encoding the protein sequences to be displayed are inserted into a coat protein gene, e.g. gene III or gene VIII of the phage. The resultant fusion proteins are displayed on the surface of the capsid. Protein VIII is present in approximately 2700 copies per phage, compared to 3 to 5 copies for protein III (Jacobsson, supra (1996)). Multivalent expression vectors, such as phagemids, can be used for manipulation of the nucleic acid sequences encoding the recombinant catalytic polypeptides and production of phage particles in bacteria (see, e.g., Felici, J. Mol. Biol. 222:301-310 (1991)).

Phagemid vectors are often employed for constructing the phage library. These vectors include the origin of DNA replication from the genome of a single-stranded filamentous bacteriophage, e.g., M13 or fl, and require the supply of the other phage proteins to create a phage. This is usually supplied by a helper phage which is less efficient at being packaged into phage particles. A phagemid can be used in the same way as an orthodox plasmid vector, but can also be used to produce filamentous bacteriophage particle that contain single-stranded copies of cloned segments of DNA.

The displayed polypeptide does not need to be a fusion protein. For example, a recombinant catalytic polypeptide may attach to a coat protein by virtue of a non-covalent interaction, e.g., a coiled coil binding interaction, such as Jun/Fos binding, or a covalent interaction mediated by cysteines (see, e.g., Crameri et al., Eur. J. Biochem. 226:53-58 (1994)) with or without additional non-covalent interactions. A display system has been described, for example, by Morphosys, where one cysteine is put at the C terminus of the single chain Fᵥ or F_{ab}, and another is put at the N terminus of g3p. The two assemble in the periplasm and display occurs without a fusion gene or protein.

The coat protein does not need to be endogenous. For example, DNA binding proteins can be incorporated into the phage/phagemid genome (see, e.g., McGregor & Robins, Anal. Biochem. 294:108-117 (2001)). When the sequence recognized by such proteins is also present in the genome, the DNA binding protein becomes incorporated into the phage/phagemid. This can serve as a display vector protein. In some cases it has been shown that incorporation of DNA binding proteins into the phage coat can occur independently of the presence of the recognized DNA signal.

Other phages can also be used. For example, T7 vectors, T4 vector, T2 vectors, or lambda vectors can be employed in which the displayed product on the mature phage particle is released by cell lysis.

(2) Other Display Libraries

In addition to phage display libraries, analogous epitope display libraries can also be used. For example, the methods of the invention can also use yeast surface displayed libraries (see, e.g., Boder, Nat. Biotechnol. 15:553-557 (1997)), which can be constructed using such vectors as the pYD 1 yeast expression vector. Other potential display systems include mammalian display vectors and E. coli libraries. For example, the E. coli flagellin protein can be used to display fluorescent binding ligand sequences.

In vitro display library formats known to those of skill in the art can also be used, e.g., ribosomal display libraries and mRNA display libraries. In these in vitro selection technologies, proteins are made using cell-free translation and physically linked to their encoding mRNA after in vitro translation. In typical methodology for generating these libraries, DNA encoding the sequences to be selected are transcribed in vitro and translated in a cell-free system.

In a ribosomal display library (see, e.g., Mattheakis et al., Proc. Natl. Acad. Sci USA 91:9022-9026 (1994); Hanes & Pluckthrun, Proc. Natl. Acad. Sci USA 94:4937-4942, (1997)) the link between the mRNA encoding the fluorescent binding ligand of the invention and the ligand is the ribosome itself. The DNA construct is designed so that no stop codon is included in the transcribed mRNA. Thus, the translating ribosome stalls at the end of the mRNA and the encoded protein is not released. The encoded protein can fold into its correct structure while attached to the ribosome. The complex of mRNA, ribosome and protein is then directly used for selection against an immobilized target. The mRNA from bound ribosomal complexes is recovered by dissociation of the complexes with EDTA and amplified by RT-PCR.

Method and libraries based on mRNA display technology, also referred to herein as puromycin display, are described, for example in U.S. Pat. Nos. 6,261,804; 6,281,223; 6,207,446; and 6,214,553. In this technology, a DNA linker attached to puromycin is first fused to the 3′end of mRNA. The polypeptide, such as the recombinant catalytic polypeptide of the present invention, is then translated in vitro and the ribosome stalls at the RNA-DNA junction. The puromycin, which mimics aminoacyl tRNA, enters the ribosomal A site and accepts the nascent polypeptide. The translated polypeptide is thus covalently linked to its encoding mRNA. The fused molecules can then be purified and screened for specific binding and proteolytic activity. The nucleic acid sequences encoding recombinant polypeptides with desired enzymatic activity can then be obtained, for example, using RT-PCR.

The recombinant catalytic polypeptides and sequences, e.g., DNA linkers, for conjugation to puromycin, can be joined by methods well known to those of skill in the art and are described, for example, in U.S. Pat. Nos. 6,261,804; 6,281,223; 6207446; and 6,214553.

Other technologies involve the use of viral proteins (e.g., protein A) that covalently attach polypeptides to the genes that encodes them. Fusion proteins are created that join the recombinant catalytic polypeptides to the protein A sequence, thereby providing a mechanism to attach these recombinant catalytic polypeptides to the genes that encode them.

Plasmid display systems may also rely on the fusion of displayed polypeptides to DNA binding proteins, such as the lac repressor (see, e.g., Gates et al., J. Mol. Biol. 255:373-386 (1996)). When the lac operator is present in the plasmid as well, the DNA binding protein binds to it and can be co-purified with the plasmid. Libraries can be created linked to the DNA binding protein, and screened upon lysis of the bacteria. The desired plasmid/polypeptide can be rescued by transfection, or amplification.

B. Screening Libraries

Methods of screening the libraries of the present invention are based on the desired characteristics of the recombinant catalytic polypeptides, i.e., their ability to specifically bind and cleave target proteins. The libraries may thus be screened for the ability of catalyzing proteolysis of target proteins, and various in vitro assays detecting enzymatic activity described in previous sections can be used. In libraries that are constructed using a display vector, such as a phage display vector, the selected clones, e.g., phage, are then used to infect bacteria.

Once a recombinant catalytic polypeptide is selected, the nucleic acid encoding the polypeptide is readily obtained. This nucleic acid sequence may then be expressed using any of a number of systems, as described in an earlier section, to obtain the desired quantities of the recombinant catalytic polypeptide.

IX. Non-Human Transgenic Mammals

A nucleic acid sequence encoding a polypeptide comprising the variable region of the human light chain of the present invention (Vₗ ), e.g., SEQ ID NO: 1, 3, 5, 7, 9, 11, 13, 15, 17, 19, 21, 23, 25, or 27, can be introduced into a non-human mammal to generate a transgenic animal that express the human V_{L}. Unlike the transgenic animal models more commonly seen, the transgene expressed by the transgenic mammals of the present invention need not replace at least one allele of the endogenous coding sequence responsible for the variable regions of antibody light chains following somatic recombination. Due to allelic exclusion, the presence of an exogenous, post-somatic rearrangement version of V_{L} DNA will inhibit the endogenous germline genes of the V_{L} loci from undergoing somatic rearrangement and contributing to the makeup of antibody light chains this mammal may produce. Thus, when exposed to a particular antigen, the mammal will generate heterologous antibodies comprising a light chain with human V_{L} (and therefore with proteolytic activity), such as SEQ ID NO:2, 4, 6, 8, 10, 12, 14, 16, 18, 20, 22, 24, 26, or 28, and a heavy chain of endogenous origin with specificity for the antigen. Such heterologous antibodies are invaluable in research and in treating certain conditions in live subjects. On the other hand, a method that directs the integration of the transgene to the locus of an endogenous allele will fully serve the purpose of practicing the present invention as well.

The general methods of generating transgenic animals have been well established and frequently practiced. The following sections provide a brief description of some of the well known techniques to generate transgenic non-human mammals for the purpose of illustration, not limitation.

A. Targeting of the Disruption: Homologous Recombination

The process of homologous recombination can be used to control the site of integration of a transgene, i.e., a nucleic acid comprising the coding sequence of the variable region of a human catalytic light chain (e.g., SEQ ID NO:1, 3, 5, 7, 9, 11, 13, 15, 17, 19, 21, 23, 25, or 27), into the location of the endogenous V_{L} coding sequence of an animal cell and thereby disrupt that gene and prevent its normal expression. Homologous recombination is described in detail by Watson in Molecular Biology of the Gene, 3rd Ed., W. A. Benjamin, Inc., Menlo Park, Calif. (1977). In brief, homologous recombination is a natural cellular process that results in the scission of two nucleic acid molecules having identical or substantially similar (i.e. “homologous”) sequences, and the ligation of the two molecules such that one region of each initially present molecule is now ligated to a region of the other initially present molecule (Sedivy, Bio-Technol., 6:1192-1196 (1988)).

Homologous recombination is exploited by a number of various methods of “gene targeting” well known to those of skill in the art (see, e.g., Mansour et al., Nature 336:348-352 (1988); Capecchi et al., Trends Genet. 5:70-76 (1989); Capecchi, Science 244:1288-1292 (1989); Capecchi et al., Current Communications in Molecular Biology, pp45-52, Capecchi, M. R. (ed.), Cold Spring Harbor Press, Cold Spring Harbor, N.Y. (1989); Frohman et al., Cell 56: 145-147 (1989)). Some approaches further involve increasing the frequency of recombination between two DNA molecules by treating the introduced DNA with agents that stimulate recombination (e.g., trimethyipsoralen, IJY light, etc.), however, most approaches utilize various combinations of selectable markers to facilitate isolation of the transformed cells. One such selection method is termed positive/negative selection (PNS) and is described by Thomas and Cappechi, Cell 51:503-5 12 (1987). Other strategies that select for homologous recombination events but do not use PNS may also be used. For example, a positive selection gene such as a bacterial drug resistance gene may be used. In some cases where a drug resistance is undesirable for a transgenic animal, one or more genetic elements can be included in the transgene/knockout construct that allows the drug resistance gene to be excised following homologous recombination. O'Gorman et al. (Science 251:1351-1355 (1991)) have described the FLP/FRT recombinase system from yeast represents as such a set of genetic elements.

The same general methods can be used to replace both alleles of the V_{L} encoding sequences. The frequency of such dual recombination events is, however, significantly lower. Animals with a single allele substitution can be cross-bred to produce homozygotes with both alleles disrupted. Further, as stated above, allelic exclusion ensures the dominance of human V_{L} in all antibody light chains produced by a transgenic animal. A double substitution is thus not necessary at the level of homologous recombination.

B. Transformation of Cells

To produce the transgenic animals of the present invention, cells are transformed with a construct containing the transgene comprising human V_{L} coding sequence, e.g., SEQ ID NO:1, 3, 5, 7,9, 11, 13, 15, 17, 19,21,23, 25, or 27. In this context, the term “transformed” is defined as introduction of exogenous DNA into a target cell by any means known to the skilled artisan. These methods of introduction include, but are not limited to, transfection, microinjection, infection (with, for example, retroviral-based vectors), electroporation, and microballistics. The term “transformed,” unless otherwise indicated, is not intended herein to indicate alterations in cell behavior and growth patterns accompanying immortalization, density-independent growth, malignant transformation or similar acquired states in culture.

To create animals having a particular gene substituted in all cells, it is preferable to introduce a transgene construct into the germ cells (sperm or eggs, i.e., the “germ line”) of the desired species. Genes or other DNA sequences can be introduced into the pronuclei of fertilized eggs by microinjection or other methods as described below. Following pronuclear fusion, the developing embryo may carry the introduced gene in all its somatic and germ cells since the zygote is the mitotic progenitor of all cells in the embryo. Since targeted insertion of a transgene construct is a relatively rare event, it is desirable to generate and screen a large number of animals when employing such an approach. Because of this, it can be advantageous to work with the large cell populations and selection criteria that are characteristic of cultured cell systems. However, for production of transgenic animals from an initial population of cultured cells, it is preferred that a cultured cell containing the desired transgene construct be capable of generating a whole animal. This is generally accomplished by placing the cell into a developing embryo environment of some sort.

Cells capable of giving rise to at least several differentiated cell types are called “pluripotent” cells. Pluripotent cells capable of giving rise to all cell types of an embryo, including germ cells, are hereinafter termed “totipotent” cells. Totipotent murine cell lines (embryonic stem, or “ES” cells), for example, have been isolated by culture of cells derived from very young embryos (blastocysts). Such cells are capable, upon incorporation into an embryo, of differentiating into all cell types, including germ cells, and can be employed to generate animals containing a transgene replacing the endogenous counterpart. Therefore, cultured ES cells can be transformed with a transgene construct, as described herein, and cells selected in which the murine V_{L} gene has been replaced by the human V_{L} gene through insertion of the transgene construct.

Several general methods of cell transformation are described as follows.

(1) Microinjection Methods

Microinjection is one preferred method for transformation of a zygote. In mouse, the male pronucleus reaches the size of approximately 20 micrometers in diameter which allows reproducible injection of 1-2 p1 of DNA solution. The use of zygotes as a target for gene transfer has a major advantage in that in most cases the injected DNA will be incorporated into the host gene before the first cleavage (Brinster et al., Proc. Natl. Acad. Sci. USA 82:4438-4442 (1985)). As a consequence, all cells of the transgenic non-human animal will carry the incorporated transgene. This will, in general, also be reflected in the efficient transmission of the transgene to offspring of the founder since 50% of the germ cells will harbor the transgene.

The human V_{L} gene, e.g., one comprising a nucleic acid sequence of SEQ ID NO: 1, 3, 5, 7, 9, 11, 13, 15, 17, 19, 21, 23, 25, or 27, being introduced by this method need not be incorporated into any kind of self-replicating plasmid or virus (Jaenisch, Science 240:1468-1474 (1988)). Once the DNA molecule has been injected into the fertilized egg, the egg is implanted into the uterus of a recipient female and allowed to develop into an animal. Since all of the animal's cells are derived from the implanted fertilized egg, all of the cells of the resulting animal (including the germ line cells) shall contain the introduced human V_{L} gene. If, as occurs in about 30% of events, the first cellular division occurs before the human V_{L} gene has integrated into the cell's genome, the resulting animal will be a chimeric animal.

By breeding and inbreeding such animals, it is possible to routinely produce heterozygous and homozygous transgenic animals. Despite any unpredictability in the formation of such transgenic animals, the animals have generally been found to be stable, and to be capable of producing offspring that retain and express the introduced human V_{L} gene.

The success rate for producing transgenic animals is greatest in mice. Approximately 25% of fertilized mouse eggs into which DNA has been injected, and which have been implanted in a female, will become transgenic mice. A number of other transgenic animals have also been produced by this method. These include rabbits, sheep, cattle, and pigs (Jaenisch Science 240:1468-1474 (1988); Hammer et al., J. Animal Sci. 63:269 (1986); Hammer et al. Nature 315:680 (1985); Wagner et al., Theriogenology 21:29 (1984)).

(2) Retroviral Methods

Retroviral infection is another means to introduce a transgene into a non-human mammal. The developing non-human embryo can be cultured in vitro to the blastocyst stage. During this time, the blastomeres can be targets for retroviral infection (Jaenich, Proc. Natl. Acad. Sci. USA 73:1260-1264 (1976)). Efficient infection of the blastomeres is obtained by enzymatic treatment to remove the zona pellucida (Hogan et al., Manipulating the Mouse Embryo, Cold Spring Harbor Laboratory Press, Cold Spring Harbor, N.Y. (1986)). The viral vector system used to introduce the transgene, i.e., the human V_{L} gene, is typically a replication-defective retrovirus carrying the transgene (Jahner et al., Proc. Natl. Acad. Sci. USA 82:6927-6931 (1985); Van der Putten et al., Proc. Natl. Acad. Sci. USA, 82:6148-6152 (1985)). Transfection is easily and efficiently obtained by culturing the blastomeres on a monolayer of virus-producing cells (Van der Putten et al., supra; Stewart et al., EMBO J., 6:383-388 (1987)). Alternatively, infection can be performed at a later stage. Virus or virus-producing cells can be injected into the blastocoele (Jahner et al., Nature 298:623-628 (1982)). Most of the founders will be mosaic for the transgene since incorporation occurs only in a subset of the cells, which formed the transgenic non-human animal. Further, the founder may contain various retroviral insertions of the transgene at different positions in the genome which generally will segregate in the offspring. In addition, it is also possible to introduce transgenes into the germ line, albeit with low efficiency, by intrauterine retroviral infection of the midgestation embryo (Jahner et al., supra).

(3) ES Cell Implantation

A third and preferred target cell for transgene introduction is the embryonic stem cell (ES). ES cells are obtained from pre-implantation embryos cultured in vitro and fused with embryos (Evans et. al., Nature 292:154-156 (1981); Bradley et al., Nature 309:255-258 (1984); Gossler et al., Proc. Natl. Acad. Sci. USA 83:9065-9069 (1986); and Robertson et al., Nature 322:445-448 (1986)). Transgenes can be efficiently introduced into the ES cells by a number of means well known to those of skill in the art. The transformed ES cells can thereafter be combined with blastocysts from a non-human animal, such as mouse. The ES cells thereafter colonize the embryo and contribute to the germ line of the resulting chimeric animal (for a review see Jaenisch Science 240:1468-1474 (1988)).

The nucleotide sequence containing the human V_{L} gene, e.g., SEQ ID NO: 1, 3, 5, 7, 9, 11, 13, 15, 17, 19, 21, 23, 25, or 27, may be introduced into the pluripotent cell by any method which will permit the introduced molecule to undergo recombination at its regions of homology. Transgenes can be efficiently introduced into the ES cells by DNA transfection or by retrovirus-mediated transduction.

The nucleic acid can be introduced, for example, by electroporation (Toneguzzo et al., Nucleic Acids Res. 16:55 15-5532 (1988); Quillet et al., J. Immunol., 141:17-20 (1988); Machy et al., Proc. Natl. Acad. Sci. USA 85:8027-8031 (1988)). After permitting the introduction of the nucleic acid containing transgene, the cells are cultured under conventional conditions, as are known in the art.

In order to facilitate the recovery of those cells that have received the nucleic acid containing the transgene, it is preferable to introduce the nucleic acid containing the transgene, e.g., a nucleic acid comprising SEQ ID NO:1, 3, 5, 7, 9, 11, 13, 15, 17, 19, 21, 23, 25, or 27, in combination with a second gene encoding a detectable marker. Preferably, the detectable marker gene will be expressed in the recipient cell and result in a selectable phenotype. Numerous selectable markers are well known to those of skill in the art. Some examples include the hprt gene (Littlefield, Science 145:709-710 (1964)), the thymidine kinase gene of herpes simplex virus (Giphart-Gassier et al., Mutat, Res., 214:223-232 (1989)), the nDtII gene (Thomas et al., Cell 51:503-512 (1987); Mansour et al., Nature 336:348-352 (1988)). The detectable marker gene may also be any gene that can compensate for a recognizable cellular deficiency.

The transgenic animal cells of the present invention are prepared by introducing one or more nucleic acids into a precursor pluripotent cell, most preferably an ES cell, or equivalent (Robertson, Current communications in Molecular Biology, pp 39-44, Capecchi, M. R. (ed.), Cold Spring Harbor Press, Cold Spring Harbor, N.Y. (1989)). The term “precursor” is intended to denote only that the pluripotent cell is a precursor to the desired (“transfected”) pluripotent cell, which is prepared in accordance with the teachings of the present invention. The pluripotent (precursor or transfected) cell may be cultured in vivo, in a manner known in the art (Evans et al., Nature 292:154-156 (1981)) to form a chimeric or transgenic animal. The transfected cell, and the cells of the embryo that it forms upon introduction into the uterus of a female are herein referred to respectively, as “embryonic stage” ancestors of the cells and animals of the present invention.

Any ES cell may be used in accordance with the present invention. It is, however, preferred to use primary isolates of ES cells. Such isolates may be obtained directly from embryos such as the CCE cell line disclosed by Robertson, E. J., Current Communications in Molecular Biology, pp. 39-44, Capecchi, M. R. (ed.), Cold Spring Harbor Press, Cold Spring Harbor, N.Y. (1989), or from the clonal isolation of ES cells from the CCE cell line (Schwartzberg et al., Science 212:799-803 (1989)). Such clonal isolation may be accomplished according to the method of Robertson, Teratocarcinomas and Embryonic Stem Cells: A Practical Approach, E. J. Robertson, Ed., IRL Press, Oxford (1987). The purpose of such clonal propagation is to obtain ES cells that have a greater efficiency for differentiating into an animal. Clonally selected ES cells are approximately 10-fold more effective in producing transgenic animals than the progenitor cell line CCE. An example of ES cell lines which have been clonally derived from embryos are the ES cell lines, AB I (hprt+) or AB2.1 (hprt−).

ES cell lines may be derived or isolated from any mammals such as rodents, rabbits, sheep, goats, fish, pigs, cattle, and primates. Cells derived from rodents (i.e. mouse, rat, hamster, etc.) are preferred. ES cell lines have been derived for mice and pigs as well as other animals (see, e.g., PCT Publication No. WO/90/03432; PCT Publication No. 94/26884). Generally these cells lines must be propagated in a medium containing a differentiation-inhibiting factor (DIF) to prevent spontaneous differentiation and loss of mitotic capability. Leukemia Inhibitory Factor (LIF) is particularly useful as a DIF. Other DIF's useful for prevention of ES cell differentiation include, without limitation, Oncostatin M (Gearing and Bruce, The New Biologist 4:61-65 (1992)), interleukin 6 (IL-6) with soluble IL-6 receptor (sIL-6R) (Taga et al., Cell 58:573-581 (1989)), and ciliary neurotropic factor (CNTF) (Conover et al., Development 19:559-565 (1993)). Other known cytokines may also function as appropriate DIP'S, alone or in combination with other DIF's.

C. Production of Transgenic Animals Via Somatic Cell Nuclear Transfer

Production of the transgenic animals of this invention is not dependent on the availability of ES cells, as these animals can be produced using methods of somatic cell nuclear transfer. For example, a somatic cell can be obtained from the species in which the native V_{L} gene is to be replaced by the human V_{L} gene (e.g., SEQ ID NO: 1, 3, 5, 7, 9, 11, 13, 15, 17, 19, 21, 23, 25, or 27)of a proteolytic light chain. The cell is first transfected with a construct that introduces the human V_{L} gene into the location of the endogenous V_{L} gene, e.g., via heterologous recombination. Cells harboring the newly introduced human V_{L} gene for a catalytic light chain are selected as described above. The nucleus of such a transformed cell is then placed in an unfertilized enucleated egg (e.g., an egg from which the natural nuclei have been removed by microsurgery). Once the transfer is complete, the recipient egg contains a complete set of genes, just as they would if they had been fertilized by sperm. The eggs are then cultured for a period before being implanted into a host mammal (of the same species that provided the egg) where they are carried to term, culminating in the berth of a transgenic animal comprising a nucleic acid construct containing one or more substituted V_{L} genes.

The production of viable cloned mammals following nuclear transfer of cultured somatic cells has been reported for a wide variety of species including, but not limited to calves (Kato et al., Science 262:2095-2098 (1998)), sheep (Campbell et al., Nature 380:64-66 (1996)), mice (Wakayama and Yanagimachi, Nat. Genet. 22:127-128 (1999)), goats (Baguisi et al., Nat. Biotechnol. 17:456-461 (1999)), monkeys (Meng et al., Biol. Reprod. 57:454-459 (1997)), and pigs (Bishop et al., Nature Biotechnol. 18:1055-1059 (2000)). Nuclear transfer methods have also been used to produce clones of transgenic animals. Thus, for example, the production of transgenic goats carrying the human antithrobin III gene by somatic cell nuclear transfer has been reported (Baguisi et al., Nature Bioiechnol. 17:456-461 (1999)).

Using methods of nuclear transfer as described in these and other references, cell nuclei derived from differentiated fetal or adult, mammalian cells are transplanted into enucleated mammalian oocytes of the same species as the donor nuclei. The nuclei are reprogrammed to direct the development of cloned embryos, which can then be transferred into recipient females to produce fetuses and offspring, or used to produce cultured inner cell mass (CICM) cells. The cloned embryos can also be combined with fertilized embryos to produce chimeric embryos, fetuses, and/or offspring.

Somatic cell nuclear transfer also allows simplification of transgenic procedures by working with a differentiated cell source that can be clonally propagated. This eliminates the need to maintain the cells in an undifferentiated state, thus, genetic modifications, both random integration and gene targeting, are more easily accomplished. Also by combining nuclear transfer with the ability to modify and select for these cells in vitro, this procedure is more efficient than previous transgenic embryo techniques.

Nuclear transfer techniques or nuclear transplantation techniques are known in the literature. See, in particular, Campbell et al., Theriogenology 43:181 -(1995); Collas et al., Mol. Report Dev. 38:264-267 (1994); Keefer et al., Biol. Reprod. 50:935-939 (1994); Sims et al., Proc. Natl. Acad. Sci. USA 90:6143-6147 (1993); WO 94/26884; WO 94/24274, WO 90/03432, U.S. Pat. Nos. 5,945,577, 4,944,384, and 5,057,420.

Differentiated mammalian cells are those cells that are past the early embryonic stage. More particularly, the differentiated cells are those from at least past the embryonic disc stage. The differentiated cells may be derived from ectoderm, mesoderm or endoderm.

Mammalian cells useful in the present invention may be obtained by well known methods. They include, by way of example, epithelial cells, neural cells, epidermal cells, keratinocytes, hematopoietic cells, melanocytes, chondrocytes, lymphocytes (B and T lymphocytes), erythrocytes, macrophages, monocytes, mononuclear cells, fibroblasts, cardiac muscle cells, and other muscle cells, etc. Moreover, the mammalian cells used for nuclear transfer may be obtained from different organs, e.g., skin, lung, pancreas, liver, stomach, intestine, heart, reproductive organs, bladder, kidney, urethra, and other urinary organs. Suitable donor cells, i.e., cells useful in the subject invention, may be obtained from any cell or organ of the body, including all somatic or germ cells.

Fibroblast cells are an ideal cell type because they can be obtained from developing fetuses and adult animals in large quantities. Fibroblast cells are differentiated somewhat and, thus, were previously considered a poor cell type to use in cloning procedures. In particular, these cells can be easily propagated in vitro with a rapid doubling time and can be clonally propagated for use in gene targeting procedures.

After substitution of the endogenous V_{L} gene with the human V_{L} gene, e.g., SEQ ID NO:1, 3, 5, 7, 9, 11, 13, 15, 17, 19, 21, 23, 25, or 27, from a catalytic light chain in a somatic cell, the nucleus of the cell is transferred into a mammalian oocyte, such as an oocyte from sheep, cows, pigs, horses, rabbits, guinea pigs, mice, hamsters, rats, or any non-human primates. Methods for oocyte isolation are well known in the art.

The oocytes are generally matured in vitro before they are used as recipient cells for nuclear transfer. This process generally involves collecting immature (prophase I) oocytes from mammalian ovaries, e.g., mouse ovaries, and maturing the oocytes in a maturation medium until the oocyte attains the metaphase II stage. This period of time is known as the “maturation period.” Various types of maturation medium are known to those skilled in the art. In addition, oocytes in metaphase II, which have been matured in vivo, have also been successfully used in nuclear transfer procedures.

After the maturation period, the oocytes will be enucleated. Enucleation may be effected by known methods, such as described in U.S. Pat. No. 4,994,384. Enucleation can also be accomplished via microsurgery, e.g., using a micropipette to remove the polar body and the adjacent cytoplasm. The oocytes can then be screened to identify those of which have been successfully enucleated. The screening can be performed by staining the oocytes with a variety of dyes that stains nucleic acids, one example of such dye is 33342 Hoechst dye.

A single mammalian cell of the same species as the enucleated oocyte are then used to produce the nuclear transfer (NT) unit according to methods known in the art. For example, the cells can be fused by electrofusion as disclosed in U.S. Pat. No. 4,997,384. Fusion can also be accomplished using Sendai virus as a fusogenic agent (Graham, Inot. Symp. Monogr. 9:19 (1969)). In some cases, especially where the donor nuclei is small, it may be preferable to inject the nucleus directly into the oocyte. See, e.g., Collas and Barnes, Mol. Reprod. Dev. 38:264-267 (1994).

Shortly after fusion, the resultant fused NT units are activated by various known methods. Such methods include, e.g., culturing the NT unit at sub-physiological temperature, in essence by applying a cold, or actually cool temperature shock to the NT unit. Activation may also be achieved by known activation methods, such as electrical and chemical shock. Suitable oocyte activation methods are the subject of U.S. Pat. No. 5,496,720.

The activated NT units can then be cultured in a suitable in vitro culture medium until the generation of CICM cells and cell colonies. Culture media suitable for culturing and maturation of embryos are well known in the art. U.S. Pat. No. 5,096,822, for example, describes such a maintenance medium.

Afterward, the cultured NT unit or units are preferably washed and then placed in a suitable media on a suitable confluent feeder layer. Suitable feeder layers include, by way of example, fibroblasts and epithelial cells, e.g., fibroblasts and uterine epithelial cells derived from murine (e.g., mouse or rat) fibroblasts. The NT units are cultured on the feeder layer until the NT units reach a size suitable for transferring to a recipient female, or for obtaining cells which may be used to produce CICM cells or cell colonies.

The methods for embryo transfer and recipient animal management for somatic cell nuclear transfer are standard procedures used by those skilled in the art. For review see, Siedel, G. E., Jr., “Critical review of embryo transfer procedures with cattle” in Fertilization and Embryonic Development in Vitro, page 323, L. Mastroianni, Jr. and J. D. Biggers, ed., Plenum Press, New York, N.Y. (1981).

### EXAMPLES

It has been known that human autoimmune diseases confer a predisposition to the production of hydrolytic antibodies. Additionally, hemophilia A patients produce inhibitors to factor VIII, some of which have been shown to be proteolytic antibodies that cleave factor VIII during replacement therapy (Lacroix-Desmazes et al, N. Engl. J. Med., 346: 662-667 (2002)). The dramatic clinical effect of these proteolytic antibodies on the course of hemophilia in these patients suggests that exogenously provided proteolytic antibodies specific for a target protein can positively effect the course of several other diseases by catalytically eliminating target proteins crucial to pathogenesis. Production of such proteolytic antibodies for therapeutic use will be enhanced if the genetic basis for such catalytic activity are understood and can be harnessed. The genetic basis for these human proteolytic antibodies has, however, not yet been elucidated. Recent work on proteolytic antibodies in mice have defined a proteolytic light chain in an antibody raised against vasoactive intestinal polypeptide (VIP). The genetic basis for the mouse proteolytic antibody provides insight into the possible mechanism by which human proteolytic antibodies may function.

Identification of Catalytic V_{L} Sequences

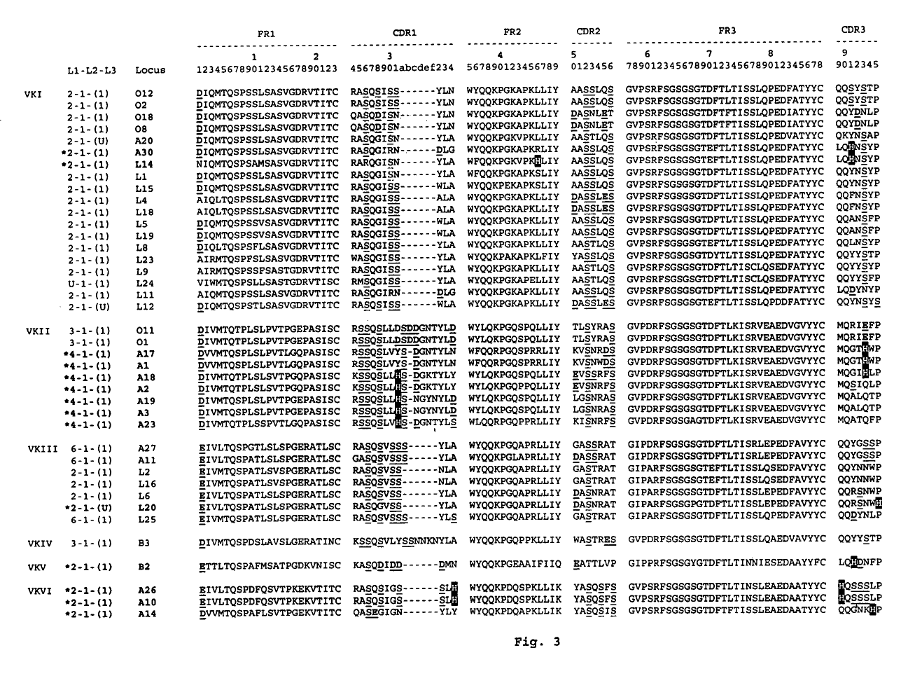

The sequence of the V region encoding mouse anti-VIP proteolytic light chain belongs to the Kappa II family of V regions. Additionally, other esterolytic antibodies share a predilection to utilize the Kappa II family, suggesting that this family contains domains important in catalysis. In order to determine the human genetic basis for proteolytic antibodies, and to harness the use of developing human therapeutic proteolytic antibodies, the human kappa repertoire was analyzed for genes containing putative serine protease triads. Several genes were identified and are illustrated in FIG. 3. These genes include A30, L14, A17, A1, A18b, A2, A19, A3, A23, L20, B2, A26, A10, and A14.

Cloning V_{L} Genes

In order to clone the genes encoding these potentially catalytic variable regions, PCR primers were designed to hybridize to the 5′ terminus of the leader region and intron, and to the 3′ recombination signal sequence. Convenient restriction sites were added to the 5′ end of each primer for subsequent cloning steps. PCR was performed using 100 ng of human genomic DNA (Clontech, Palo Alto, Calif.). The 50 μl reaction was started by heating the sample to 94° C. for 5 minutes, adding 1 μl pfu polymerase (Stratagene, La Jolla, Calif.) followed by an additional 5 minutes at 94° C. PCR was performed for 40 cycles with annealing at 56° C. for 30 seconds, extending at 70° C. for 30 seconds, and denaturing at 94° C. for 20 seconds. A final extension was done at 70° C. for 5 minutes. A 1.5% agarose gel resolved 10 μl of the PCR reaction, and the product formed was seen to migrate near the expected size of 300 bp. Using overlap extension method, the V-regions were fused in frame to human J Kappal using a 5′ primer for the V region, a 3′ primer encompassing all of J Kappal and a “bridging” primer that overlapped the V and J regions. PCR was done according to the cycling conditions above. Successful construction of the V-J hybrid was confirmed by resolving the 150 bp PCR product on a 1.5% agarose gel, and by DNA sequencing.

analysis of catalytic Function

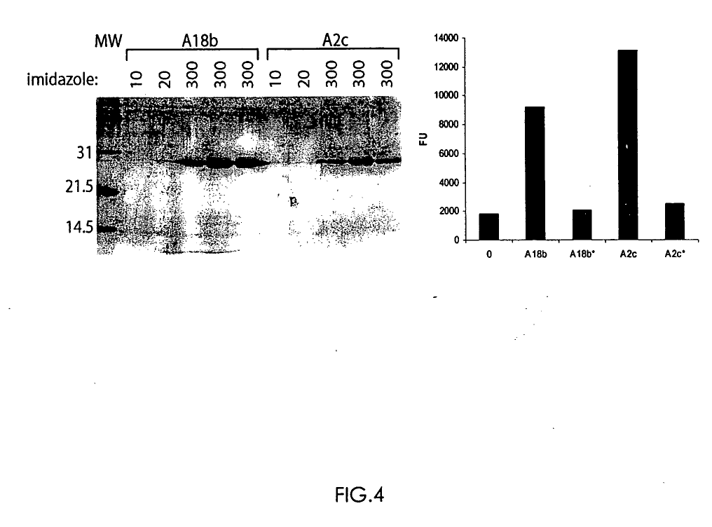

To determine the catalytic function of the human light chains, SEQ ID NOs: 1, 3, 5, 7, 9, 11, 13, 15, 17, 19, 21, 23, 25, and 27 are each fused to a human CL encoding sequence, e.g., a kappa CL gene, and then cloned into an expression vector containing the CMV promotor and a VH4 leader sequence and transfected into nonsecreting myeloma cell lines, such as NSO or SP2/0. Supernatants from the transfectants are removed and analyzed for proteolytic activity against a peptide-MCA substrate (Sigma, St. Louis, Mo.). The supernatants are incubated in 60 μl of 50 mM Tris-HCl, 100 mM glycine, and 0.025% Tween 20, pH 7.7 in white 96 well plates with varying concentrations of peptide-MCA. Supernatants from non-transfected cells are also analyzed and used as background. Hydrolysis of the peptide-MCA substrate is determined as the fluorescence of the aminomethylcoumarin leaving group (excitation 370 nm, emission 460 nm), with the concentration being determined by the simultaneous analysis of different concentrations of aminomethylcoumarin measured in the same volume in different wells. Results of the purification of A18b and A2c, and catalytic assays are shown in FIG. 4.

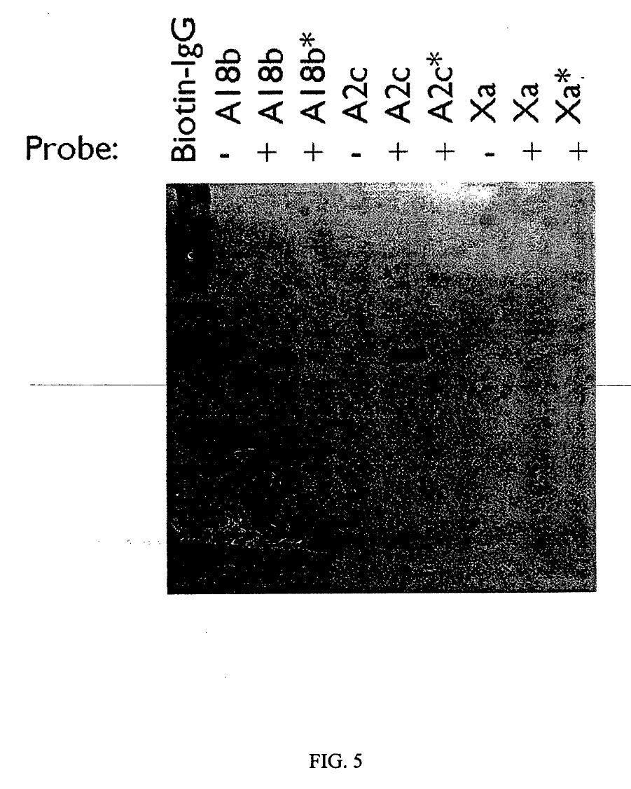

The ability of A18b and A2c to bind to a protease inhibitor probe was also undertaken. A18b and A2c were expressed in E. coli periplasm fused to a C-terminal 6-histidine tag. Antibodies were purified over immobilized nickel affinity columns according to the manufacturers instructions (Invitrogen, Carlsbad, Calif.). Biotinylated fluorophosphonate probe (10 μM) was added to 100 ng antibody light chain for 5 minutes at room temperature, then quenched with 2× SDS-PAGE loading buffer and heated to 94° C. for 3 minutes. The mixture was run on a 15% SDS-PAGE gel, transferred to a nylon membrane, blocked for 45 minutes with 3% bovine serum albumin, and incubated with streptavidin conjugated alkaline phosphatase for 1 hour. The membrane was developed with NBT/BCIP reagent. Identification of covalently binding antibody is illustrated in FIG. 5.

Operational Joining of Heterologous Heavy Chains

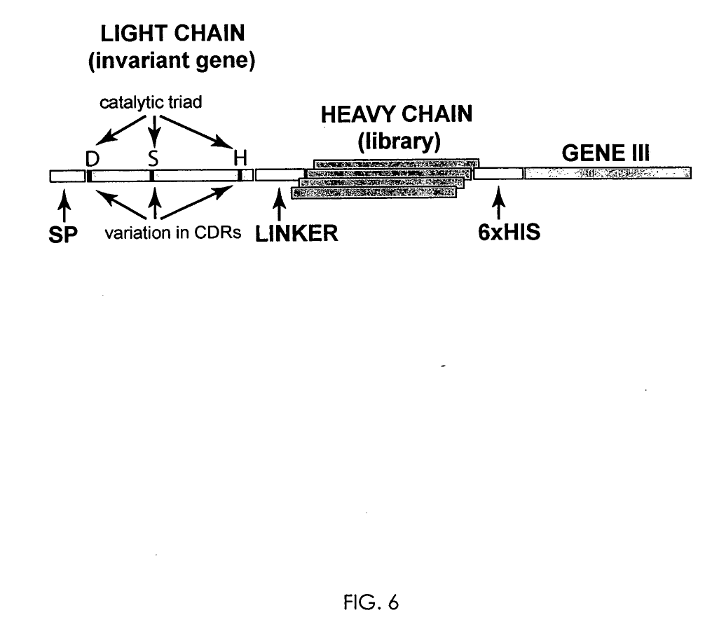

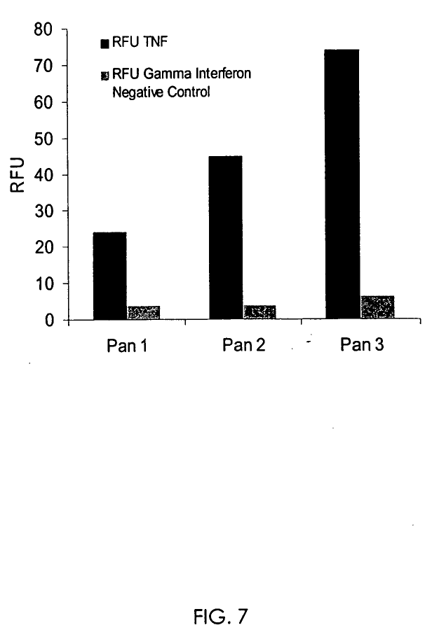

A standard phage display protocol was used to identify anti TNFα binding scFvs as follows: ˜1×10¹¹ phage from a scFv phage library (complexity 4×10¹⁰) were panned against 1 μg/well of TNFα in PBS for 2 rounds followed by a third round using a reduced antigen concentration of 0.1 ug/well TNFα. Wash stringencies were increased with each subsequent pan, with the final pan condition being 20 washes with 0.5% Tween 20/TBS. Antigen specific phage were recovered from all pans by eluting with a 2-10 fold excess of TNFα (10-20 μg/ml). Phage which did not elute with TNFα after 16 hr, were recovered by treating the wells with 100 mM glycine, pH 2.2 for 7 minutes and neutralized with 1/10 volume of 1.5 M Tris-Cl, pH 8.5 prior to storage and titering. The number of phage recovered for each pan was assayed by titering, prior to amplification and production of high titer phage stocks for future panning rounds. Pannings were only done using newly made phage preparations. Negative controls for binding specificity include interferon-γ and a phage ELISA using these controls and the phage eluted from each round is shown in FIG. 7. The heavy chains were then amplified using a standard PCR reaction (described above) and inserted into the expression vector shown in FIG. 6, thus operably joining the V_{L} containing a catalytic triad with anti-TNFα heavy chains.

To generate in a eukaryotic cell a proteolytic antibody that is specific for a given antigen such as VEGF, the light chain genes cloned above, e.g., SEQ ID NO:1, 3, 5, 7, 9, 11, 13, 15, 17, 19, 21, 23, 25, or 27, fused to a CL gene, are co-transfected into non-secreting myeloma cells NSO with a heavy chain gene encoding an anti-VEGF antibody heavy chain. Supernatants containing IgG from the transfectants are purified over a protein-A spharose column and eluted. Specific proteolytic activity is analyzed against recombinant human VEGF (commercially available from, e.g., Abcam, Cambridge, United Kingdom, or PanVera, Madison, Wis.) using HPLC. Cleavage of VEGF is evidenced by multiple peaks, representing different retention times of the different hydrolyzed products, on the HPLC column. Specificity toward VEGF is shown by the lack of such multiple peaks in a negative control, which is an identical reaction where VEGF is substituted with an irrelevant protein such as BSA or lysozyme.

What is claimed is:

1. A recombinant catalytic polypeptide for cleaving a target protein comprising a human antibody light chain operably joined to a heterologous antibody heavy chain where the light chain has a serine protease dyad and endopeptidase activity and where the heavy chain has a predetermined specificity for the target protein.

2. The recombinant catalytic polypeptide of claim 1, wherein the target protein is selected from a group consisting of growth factors, cell surface receptors, cytokines, and immunoglobulins.

3. The recombinant catalytic polypeptide of claim 2, wherein the target protein is a vascular endothelial growth factor.

4. The recombinant catalytic polypeptide of claim 2, wherein the target protein is interferon y.

5. The recombinant catalytic polypeptide of claim 2, wherein the target protein is TNF a.

6. The recombinant catalytic polypeptide of claim 2, wherein the target protein is a member of the IgE family.

7. The recombinant catalytic polypeptide of claim 2, wherein the target protein is a member of the EGF receptor family.

8. The recombinant catalytic polypeptide of claim 2, wherein the target protein is CD20.

9. The recombinant catalytic polypeptide of claim 1, wherein the human antibody light chain has a serine protease triad.

10. The recombinant catalytic polypeptide of claim 1, wherein the recombinant catalytic polypeptide is a single polypeptide chain that contains the human antibody light chain and the antibody heavy chain.

11. The recombinant catalytic polypeptide of claim 1, wherein the human antibody light chain comprises an amino acid sequence that has at least 80% identity to SEQ ID NO:2,4, 6, 8, 10,12, 14,16, 18, 20, 22,24,26, or 28.

12. The recombinant catalytic polypeptide of claim 1, wherein the human antibody light chain comprises an amino acid sequence that has at least 95% identity to SEQ ID NO:2, 4, 6, 8, 10, 12, 14, 16, 18, 20, 22, 24, 26, or 28.

13. The recombinant catalytic polypeptide of claim 1, wherein the human antibody light chain comprises an amino acid sequence of SEQ ID NO:2, 4, 6, 8, 10, 12, 14, 16, 18,20,22,24, 26, or 28.

14. A method for cleaving a target protein comprising:
contacting the target protein with a recombinant catalytic polypeptide comprising a human antibody light chain operably joined to a heterologous antibody heavy chain where the light chain has a serine protease dyad and endopeptidase activity, and where the heavy chain has a predetermined specificity for the target protein, wherein the conditions of contact are suitable to cleave the target protein.

15. The method of claim 14, wherein the target protein is selected from a group consisting of growth factors, cell surface receptors, cytokines, and immunoglobulins.

16. The method of claim 14, wherein the human antibody light chain has a serine protease triad.

17. The method of claim 14, wherein the recombinant catalytic polypeptide is a single polypeptide chain that contains the human antibody light chain and the antibody heavy chain.

18. The method of claim 14, wherein the human antibody light chain comprises an amino acid sequence that has at least 80% identity to SEQ ID NO:2, 4, 6, 8, 10, 12, 14, 16, 18, 20, 22, 24, 26, or 28.

19. The method of claim 14, wherein the human antibody light chain comprises an amino acid sequence that has at least 95% identity to SEQ ID NO:2, 4, 6, 8, 10, 12, 14, 16, 18, 20, 22, 24, 26, or 28.

20. The method of claim 14, wherein the human antibody light chain comprises an amino acid sequence of SEQ ID NO:2, 4, 6, 8, 10, 12, 14, 16, 18, 20, 22, 24, 26, or 28.

21. A method for altering the enzymatic activity of a recombinant catalytic polypeptide for cleaving a target protein, where the method comprises the steps of: mutating at least one of the CDRs of an antibody heavy chain and determining mutations that altered in enzymatic activity of the polypeptide, wherein the recombinant catalytic polypeptide comprises a human antibody light chain operably joined to a heterologous antibody heavy chain where the light chain has a serine protease dyad and endopeptidase activity, and where the heavy chain has a predetermined specificity for the target protein.

22. The method of claim 21, wherein an exonuclease is used in the step of mutating at least one of the CDRs of the antibody heavy chain.

23. The method of claim 21, wherein the target protein is selected from a group consisting of growth factors, cell surface receptors, cytokines, and immunoglobulins.

24. The method of claim 21, wherein the human antibody light chain has a serine protease triad.

25. The method of claim 21, wherein the recombinant catalytic polypeptide is a single polypeptide chain that contains the human antibody light chain and the antibody heavy chain.

26. The method of claim 21, wherein the human antibody light chain comprises an amino acid sequence that has at least 80% identity to SEQ ID NO:2, 4, 6, 8, 10, 12, 14,16, 18, 20, 22, 24, 26, or 28.

27. The method of claim 21, wherein the human antibody light chain comprises an amino acid sequence that has at least 95% identity to SEQ ID NO:2, 4, 6, 8, 10, 12, 14, 16, 18, 20, 22, 24, 26, or 28.

28. The method of claim 21, wherein the human antibody light chain comprises an amino acid sequence of SEQ ID NO:2, 4, 6, 8, 10, 12, 14, 16, 18, 20, 22, 24, 26, or 28.

29. A library of recombinant catalytic polypeptide members for cleaving target proteins, said library members comprising:
recombinant catalytic polypeptides, each comprising a human antibody light chain operably joined to a heterologous antibody heavy chain, where the light chain has a serine protease dyad and endopeptidase activity, and where the heavy chain has a specificity for a target protein, and where the members have different CDRs in their respective heavy chains.

30. The library of claim 29, wherein the target protein is selected from a group consisting of growth factors, cell surface receptors, cytokines, and immunoglobulins.

31. The library of claim 29, wherein the human antibody light chain has a serine protease triad.

32. The library of claim 29, wherein the recombinant catalytic polypeptide is a single polypeptide chain that contains the human antibody light chain and the antibody heavy chain.

33. The library of claim 29, wherein the human antibody light chain comprises an amino acid sequence that has at least 80% identity to SEQ ID NO:2, 4, 6, 8, 10, 12, 14, 16, 18, 20, 22, 24, 26, or 28.

34. The library of claim 29, wherein the human antibody light chain comprises an amino acid sequence that has at least 95% identity to SEQ ID NO:2, 4, 6, 8, 10, 12, 14, 16, 18, 20, 22, 24, 26, or 28.

35. The library of claim 29, wherein the human antibody light chain comprises an amino acid sequence of SEQ ID NO:2, 4, 6, 8, 10, 12, 14, 16, 18, 20, 22, 24, 26, or 28.

36. The library of claim 29, which is a phage display library.

37. The library of claim 29, which is a ribosomal display library.

38. The library of claim 29, which is an mRNA display library.

39. A method for cleaving a target protein in a mammal by administration of a recombinant catalytic polypeptide comprising a human antibody light chain operably joined to a heterologous antibody heavy chain where the light chain has a serine protease dyad and endopeptidase activity, and where the heavy chain has a predetermined specificity for the target protein, wherein the recombinant catalytic polypeptide is administered in an amount sufficient to lower the concentration of the target protein in the mammal.

40. The method of claim 39, wherein the target protein is selected from a group consisting of growth factors, cell surface receptors, cytokines, and immunoglobulins.

41. The method of claim 39, wherein the human antibody light chain has a serine protease triad.

42. The method of claim 39, wherein the recombinant catalytic polypeptide is a single polypeptide chain that contains the human antibody light chain and the antibody heavy chain.

43. The method of claim 39, wherein the human antibody light chain comprises an amino acid sequence that has at least 80% identity to SEQ ID NO:2, 4, 6, 8, 10, 12, 14, 16, 18, 20, 22, 24, 26, or 28.

44. The method of claim 39, wherein the human antibody light chain comprises an amino acid sequence that has at least 95% identity to SEQ ID NO:2, 4, 6, 8, 10, 12, 14, 16, 18, 20, 22, 24, 26, or 28.

45. The method of claim 39, wherein the human antibody light chain comprises an amino acid sequence of SEQ ID NO:2, 4, 6, 8, 10, 12, 14, 16, 18, 20, 22, 24, 26, or 28.

46. A nucleic acid encoding a recombinant catalytic polypeptide for cleaving a target protein comprising a human antibody light chain operably joined to a heterologous antibody heavy chain where the light chain has a serine protease dyad and endopeptidase activity, and where the heavy chain has a predetermined specificity for the target protein.

47. The nucleic acid of claim 46, wherein the target protein is selected from a group consisting of growth factors, cell surface receptors, cytokines, and immunoglobulins.

48. The nucleic acid of claim 46, wherein the human antibody light chain has a serine protease triad.

49. The nucleic acid of claim 46, wherein the recombinant catalytic polypeptide is a single polypeptide chain that contains the human antibody light chain and the antibody heavy chain.

50. The nucleic acid of claim 46, wherein the human antibody light chain comprises an amino acid sequence that has at least 80% identity to SEQ ID NO:2, 4, 6, 8, 10, 12, 14, 16, 18, 20, 22, 24, 26, or 28.

51. The nucleic acid of claim 46, wherein the human antibody light chain comprises an amino acid sequence that has at least 95% identity to SEQ ID NO:2, 4, 6, 8, 10, 12, 14, 16, 18, 20, 22, 24, 26, or 28.

52. The nucleic acid of claim 46, wherein the human antibody light chain comprises an amino acid sequence of SEQ ID NO:2, 4, 6, 8, 10, 12, 14, 16, 18, 20, 22, 24, 26, or 28.

53. A cell hosting a nucleic acid encoding a recombinant catalytic polypeptide for cleaving a target protein comprising a human antibody light chain operably joined to a heterologous antibody heavy chain where the light chain has a serine protease dyad and endopeptidase activity, and where the heavy chain has a predetermined specificity for the target protein.

54. The cell of claim 53, wherein the target protein is selected from a group consisting of growth factors, cell surface receptors, cytokines, and immunoglobulins.

55. The cell of claim 53, wherein the human antibody light chain has a serine protease triad.

56. The cell of claim 53, wherein the recombinant catalytic polypeptide is a single polypeptide chain that contains the human antibody light chain and the antibody heavy chain.

57. The cell of claim 53, wherein the human antibody light chain comprises an amino acid sequence that has at least 80% identity to SEQ ID NO:2, 4, 6, 8, 10, 12, 14, 16, 18, 20, 22, 24, 26, or 28.

58. The cell of claim 53, wherein the human antibody light chain comprises an amino acid sequence that has at least 95% identity to SEQ ID NO:2, 4, 6, 8, 10, 12, 14, 16, 18, 20, 22, 24, 26, or 28.

59. The cell of claim 53, wherein the human antibody light chain comprises an amino acid sequence of SEQ ID NO:2, 4, 6, 8, 10, 12, 14, 16, 18, 20, 22, 24, 26, or 28.

60. An isolated polypeptide that has a serine protease dyad and endopeptidase activity, and said polypeptide comprises an amino acid sequence with at least 80% identity to SEQ ID NO:2, 4, 6, 8, 10, 12, 14, 16, 18, 20, 22, 24, 26, or 28.

61. The isolated polypeptide of claim 60, wherein the polypeptide comprises an amino acid sequence with at least 95% identity to SEQ ID NO:2, 4, 6, 8, 10, 12, 14, 16, 18, 20, 22, 24, 26, or 28.

62. The isolated polypeptide of claim 60, wherein the polypeptide comprises an amino acid sequence of SEQ ID NO:2, 4, 6, 8, 10, 12, 14, 16, 18, 20, 22, 24, 26, or 28.

63. The isolated polypeptide of claim 60, wherein the polypeptide has a serine protease triad.

64. A nucleic acid encoding a polypeptide that has a serine protease dyad and endopeptidase activity, and said polypeptide comprises an amino acid sequence with at least 80% identity to SEQ ID NO:2, 4, 6, 8, 10, 12, 14, 16, 18, 20, 22, 24, 26, or 28.

65. The nucleic acid of claim 64, which encodes a polypeptide that comprises an amino acid sequence with at least 95% identity to SEQ ID NO:2, 4, 6, 8, 10, 12, 14, 16, 18, 20, 22, 24, 26, or 28.

66. The nucleic acid of claim 64, which encodes a polypeptide that comprises the amino acid sequence of SEQ ID NO:2, 4, 6, 8, 10, 12, 14, 16, 18, 20, 22, 24, 26, or 28.

67. The nucleic acid of claim 64, which comprises a nucleotide sequence of SEQ ID NO:1, 3, 5, 7, 9, 11, 13, 15, 17, 19, 21, 23, 25, or 27.

68. The nucleic acid of claim 64, which encodes a polypeptide that has a serine protease triad.

69. A cell hosting a nucleic acid encoding a polypeptide that has a serine protease dyad and endopeptidase activity, and said polypeptide comprises an amino acid sequence with at least 80% identity to SEQ ID NO:2, 4, 6, 8, 10, 12, 14, 16, 18, 20, 22, 24, 26, or 28.

70. The cell of claim 69, wherein the polypeptide comprises an amino acid sequence with at least 95% identity to SEQ ID NO:2, 4, 6, 8, 10, 12, 14, 16, 18, 20, 22, 24, 26, or 28.

71. The cell of claim 69, wherein the polypeptide comprises the amino acid of SEQ ID NO:2, 4, 6, 8, 10, 12, 14, 16, 18, 20, 22, 24, 26, or 28.

72. The cell of claim 68, wherein the nucleic acid comprises a nucleotide sequence of SEQ ID NO: 1. 3. or 5.

73. The cell of claim 69, wherein the polypeptide has a serine protease triad.

74. A transgenic non-human mammal, which expresses a transgene comprising a nucleic acid encoding a V_{L} polypeptide that has a serine protease dyad and endopeptidase activity.

75. The mammal of claim 74, wherein the polypeptide comprises an amino acid sequence with at least 80% identity to SEQ ID NO:2, 4, 6, 8, 10, 12, 14, 16, 18, 20, 22, 24, 26, or 28.

76. The mammal of claim 74, wherein the polypeptide comprises an amino acid sequence with at least 95% identity to SEQ ID NO:2, 4, 6, 8, 10, 12, 14, 16, 18, 20, 22, 24, 26, or 28.

77. The mammal of claim 74, wherein the polypeptide comprises an amino acid sequence of SEQ ID NO:2, 4, 6, 8, 10, 12, 14, 16, 18, 20, 22, 24, 26, or 28.

78. The mammal of claim 74, wherein the nucleic acid comprises a nucleotide sequence of SEQ ID NO: 1, 3, 5, 7, 9, 11, 13, 15, 17, 19, 21, 23, 25, or 27.

79. The mammal of claim 74, wherein the polypeptide has a serine protease triad.

80. The mammal of claim 74, which is a rodent.
